(19) United States
(12) Patent Application Publication
(10) Pub. No.: US 2025/0201395 A1
(43) Pub. Date: Jun. 19, 2025

(54) DYNAMICALLY OPTIMIZED SCHEDULER FOR DIALYSIS MANAGEMENT

(71) Applicant: Centient Software LLC, Traverse City, MI (US)

(72) Inventors: James E. Kemler, Glen Arbor, MI (US); John William Stanifer, Traverse City, MI (US); Jesse J. Raleigh, Petoskey, MI (US)

(21) Appl. No.: 18/980,353

(22) Filed: Dec. 13, 2024

Related U.S. Application Data
(60) Provisional application No. 63/609,944, filed on Dec. 14, 2023.

(51) Int. Cl. G16H 40/20; G06Q 10/0635; G06Q 10/1093; G16H 10/60; G16H 40/63

(52) U.S. Cl. CPC G16H 40/20; G06Q 10/0635; G06Q 10/1097; G16H 10/60; G16H 40/63

(57) ABSTRACT

A system and method for receiving a first input indicating a frequency and a duration of dialysis prescribed for a patient in a predefined time window, receiving a second input indicating an availability of one or more dialysis machines at a location and within the predefined time window, and receiving a third input indicating an availability of one or more staff at the location and within the predefined time window. The system and method may further determine one or more dates and time periods within the one or more dates in which the patient could receive dialysis based on the frequency and the duration of dialysis, wherein the determined one or more dates and the time periods are aligned with the availability of the one or more dialysis machines and the availability of the one or more staff.

### CROSS-REFERENCE TO RELATED APPLICATION

This application claims the priority benefit of U.S. Provisional Application No. 63/609,944, filed on Dec. 14, 2023, the disclosure of which is herein incorporated by reference in its entirety.

### TECHNICAL FIELD

This disclosure relates generally to the field of scheduling medical care, and more specifically, to assessing schedules for patients, care providers, and dialysis equipment.

### BACKGROUND

Conventional dialysis scheduling may include scheduling a user for a number of hours per week over a number of weeks. Such a schedule may be fixed to ensure that the patient receives time regulated treatment. The schedule is largely dictated by the needs of a treatment facility, regardless of the user's work schedule, life events, or personal preferences. As such, patients frequently miss dialysis appointments, leading to increased hospitalization and greater mortality.

### SUMMARY

Described herein are systems, devices, and methods for analyzing and generating schedules for medical appointments that may include medical equipment, medical personnel, and patients. In some aspects, the techniques described herein relate to a computer-implemented method for optimizing dialysis machine usage, the method including: receiving a first input indicating a frequency and a duration of dialysis prescribed for a patient in a predefined time window; receiving a second input indicating an availability of one or more dialysis machines at a location and within the predefined time window; receiving a third input indicating an availability of one or more staff (e.g., clinicians, administrative personnel, etc.) at the location and within the predefined time window; determining one or more dates and time periods within the one or more dates in which the patient could receive dialysis based on the frequency and the duration of dialysis, wherein the determined one or more dates and the time periods are aligned with the availability of the one or more dialysis machines and the availability of the one or more staff; and outputting an indication of the one or more dates and the time periods.

In some aspects, the techniques described herein relate to a computer-implemented method, further including: receiving a fourth input indicating one or more of: a drive time for the patient, a weather input of the location, and a delay risk metric; and automatically updating the one or more dates and the time periods based on the fourth input.

In some aspects, the techniques described herein relate to a computer-implemented method, wherein the delay risk metric is calculated based on one or more of: traffic data, a historical appointment attendance for the patient, a schedule of one or more additional dialysis machines at the location, or a schedule of one or more additional staff at the location.

In some aspects, the techniques described herein relate to a computer-implemented method, wherein the outputting includes transmitting a message to the patient. In some aspects, the techniques described herein relate to a computer-implemented method, further including receiving a selection input indicating a first time period of the time periods on a first date of the one or more dates.

In some aspects, the techniques described herein relate to a computer-implemented method, further including: receiving a cancellation input indicating cancellation of the first time period on the first date; and outputting a health impact message when a second time period on a second date within the predefined time window is not selected. In some aspects, the techniques described herein relate to a computer-implemented method, further including: receiving a cancellation input indicating cancellation of the first time period on the first date; and outputting a second time period on a second date within the predefined time window.

In some aspects, the techniques described herein relate to a computer-implemented method, further including transmitting a message to a second patient having a lower hospitalization risk than the patient, wherein the message provides alternative appointment dates for the second patient to accommodate the second time period on the second date for the patient.

In some aspects, the techniques described herein relate to a computer-implemented method, further including receiving a fourth input indicating one or both of: a fluid load or a lifestyle metric of the patient; and automatically updating the one or more dates and the time periods based on the fourth input.

In some aspects, the techniques described herein relate to a computer-implemented method, wherein the fourth input is received from a wearable device. In some aspects, the techniques described herein relate to a computer-implemented method, wherein the fluid load is based on one or both of: a historical weight gain of the patient or a historical fluid removal rate during dialysis of the patient.

In some aspects, the techniques described herein relate to a computer-implemented method, further including: receiving, from an internet-connected scale, a weight of the patient over time; and determining the historical weight gain of the patient based on the received weight. In some aspects, the techniques described herein relate to a computer-implemented method, further including: receiving a fourth input indicating a volume of fluid removed from the patient during a previous time period on a previous date; and automatically updating the one or more dates and the time periods based on the fourth input.

In some aspects, the techniques described herein relate to a computer-implemented method, wherein the patient includes a plurality of patients, each patient having an individualized frequency and duration of dialysis in an individualized, predefined time window. In some aspects, the techniques described herein relate to a computer-implemented method, wherein the location includes a plurality of locations, such that the one or more dialysis machines include a plurality of dialysis machines and the one or more staff include a plurality of technicians.

In some aspects, the techniques described herein relate to a computer-implemented method, wherein the determining further includes determining the one or more dates at one of the plurality of locations and the time periods within the one or more dates in which the patient is recommended to receive dialysis based on the frequency and the duration of dialysis. In some aspects, the techniques described herein relate to a computer-implemented method, wherein the determining is performed by a machine learning algorithm. In some aspects, the techniques described herein relate to a computer-implemented method, further including determining the availability of the one or more staff based on a schedule for each staff and one or more predefined staffing ratios.

In some aspects, the techniques described herein relate to a computer-implemented method, wherein the method is HIPPA compliant. In some aspects, the techniques described herein relate to a computer-implemented method, further including: transmitting the one or more dates and the time periods to a scheduling system of one or more physicians for treating a secondary condition of the patient; and determining an overlap between a schedule of the one or more physicians and the one or more dates and the time periods; and scheduling an appointment with the one or more physicians during the one or more dates and the time periods.

In some aspects, the techniques described herein relate to a computer-implemented method, wherein the appointment is a virtual appointment between the patient and the one or more physicians during the dialysis machine usage by the patient. In some aspects, the techniques described herein relate to a computer-implemented method, wherein the appointment is an in-person appointment between the patient and the one or more physicians during the dialysis machine usage by the patient.

In some aspects, the techniques described herein relate to a computer-implemented method, further including: receiving a fourth input including historical data including appointment frequency per time period for a physician; and automatically updating the one or more dates and the time periods to fall within a window of higher appointment frequency for the physician.

In some aspects, the techniques described herein relate to a system for optimizing dialysis machine usage, the system including: at least one processor; and memory storing instructions that, when executed by the at least one processor, cause the system to execute instructions including: receiving a first input indicating a frequency and a duration of dialysis prescribed for a patient in a predefined time window; receiving a second input indicating an availability of one or more dialysis machines at a location and within the predefined time window; receiving a third input indicating an availability of one or more staff at the location and within the predefined time window; determining one or more dates and time periods within the one or more dates in which the patient could receive dialysis based on the frequency and the duration of dialysis, wherein the determined one or more dates and the time periods are aligned with the availability of the one or more dialysis machines and the availability of the one or more staff; and outputting an indication of the one or more dates and the time periods.

In some aspects, the techniques described herein relate to a system, wherein the instructions further include: receiving a fourth input indicating one or more of: a drive time for the patient, a weather input of the location, and a delay risk metric; and automatically updating the one or more dates and the time periods based on the fourth input.

In some aspects, the techniques described herein relate to a system, wherein the outputting includes transmitting a message to the patient. In some aspects, the techniques described herein relate to a system, wherein the instructions further include receiving a selection input indicating a first time period of the time periods on a first date of the one or more dates. In some aspects, the techniques described herein relate to a system, wherein the instructions further include receiving a fourth input indicating one or both of: a fluid load or a lifestyle metric of the patient; and automatically updating the one or more dates and the time periods based on the fourth input.

In some aspects, the techniques described herein relate to a system, wherein the fourth input is received from a wearable device. In some aspects, the techniques described herein relate to a system, wherein the fluid load is based on one or both of: a historical weight gain of the patient or a historical fluid removal rate during dialysis of the patient. In some aspects, the techniques described herein relate to a system, wherein the instructions further include: receiving a fourth input indicating a volume of fluid removed from the patient during a previous time period on a previous date; and automatically updating the one or more dates and the time periods based on the fourth input.

In some aspects, the techniques described herein relate to a system, wherein the instructions further include determining the availability of the one or more staff based on a schedule for each staff and one or more predefined staffing ratios. In some aspects, the techniques described herein relate to a system, wherein the instructions further include: transmitting the one or more dates and the time periods to a scheduling system of one or more physicians for treating a secondary condition of the patient; and determining an overlap between a schedule of the one or more physicians and the one or more dates and the time periods; and scheduling an appointment with the one or more physicians during the one or more dates and the time periods.

In some aspects, the techniques described herein relate to a system, wherein the appointment is a virtual appointment between the patient and the one or more physicians during the dialysis machine usage by the patient. In some aspects, the techniques described herein relate to a system, wherein the appointment is an in-person appointment between the patient and the one or more physicians during the dialysis machine usage by the patient.

In some aspects, the techniques described herein relate to a system, wherein the instructions further include: receiving a fourth input including historical data including appointment frequency per time period for a physician; and automatically updating the one or more dates and the time periods to fall within a window of higher appointment frequency for the physician.

In some aspects, the techniques described herein relate to a computer-readable storage medium storing instructions that, when executed by a computer cause the computer to perform instructions to optimize dialysis machine usage, the instructions including: receiving a first input indicating a frequency and a duration of dialysis prescribed for a patient in a predefined time window; receiving a second input indicating an availability of one or more dialysis machines at a location and within the predefined time window; receiving a third input indicating an availability of one or more staff at the location and within the predefined time window; determining one or more dates and time periods within the one or more dates in which the patient could receive dialysis based on the frequency and the duration of dialysis, wherein the determined one or more dates and the time periods are aligned with the availability of the one or more dialysis machines and the availability of the one or more staff; and outputting an indication of the one or more dates and the time periods.

In some aspects, the techniques described herein relate to a computer-readable storage medium wherein the instructions further include: receiving a fourth input indicating one or more of: a drive time for the patient, a weather input of the location, and a delay risk metric; and automatically updating the one or more dates and the time periods based on the fourth input.

In some aspects, the techniques described herein relate to a computer-readable storage medium, wherein the outputting includes transmitting a message to the patient. In some aspects, the techniques described herein relate to a computer-readable storage medium, wherein the instructions further include receiving a selection input indicating a first time period of the time periods on a first date of the one or more dates. In some aspects, the techniques described herein relate to a computer-readable storage medium, wherein the instructions further include receiving a fourth input indicating one or both of: a fluid load or a lifestyle metric of the patient; and automatically updating the one or more dates and the time periods based on the fourth input.

In some aspects, the techniques described herein relate to a computer-readable storage medium, wherein the fourth input is received from a wearable device. In some aspects, the techniques described herein relate to a computer-readable storage medium, wherein the fluid load is based on one or both of: a historical weight gain of the patient or a historical fluid removal rate during dialysis of the patient.

In some aspects, the techniques described herein relate to a computer-readable storage medium, wherein the instructions further include: receiving a fourth input indicating a volume of fluid removed from the patient during a previous time period on a previous date; and automatically updating the one or more dates and the time periods based on the fourth input. In some aspects, the techniques described herein relate to a computer-readable storage medium, wherein the instructions further include determining the availability of the one or more staff based on a schedule for each staff and one or more predefined staffing ratios.

In some aspects, the techniques described herein relate to a computer-readable storage medium, wherein the instructions further include: transmitting the one or more dates and the time periods to a scheduling system of one or more physicians for treating a secondary condition of the patient; and determining an overlap between a schedule of the one or more physicians and the one or more dates and the time periods; and scheduling an appointment with the one or more physicians during the one or more dates and the time periods.

In some aspects, the techniques described herein relate to a computer-readable storage medium, wherein the instructions further include: receiving a fourth input including historical data including appointment frequency per time period for a physician; and automatically updating the one or more dates and the time periods to fall within a window of higher appointment frequency for the physician.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various implementations, with reference made to the accompanying drawings.

FIG. 1A illustrates an example high level system diagram for generating dynamically optimized schedules for dialysis management.

FIG. 1B illustrates an example system diagram for generating dynamically optimized schedules for dialysis management.

FIG. 2A is an example schedule generated by the system of FIGS. 1A-1B.

FIG. 2B is another example schedule generated by the system of FIGS. 1A-1B.

FIG. 3A is an example user interface generated by the system of FIGS. 1A-1B.

FIG. 3B is an example user interface generated by the system of FIGS. 1A-1B.

FIG. 4 is another example user interface generated by the system of FIGS. 1A-1B.

FIG. 5 illustrates a flow diagram of an example process for rescheduling patients.

FIG. 6 is a flowchart of an example process for generating scheduling options that optimize dialysis machine usage.

FIG. 7 is an example appointment selection process for use with the system of FIGS. 1A-1B.

FIG. 8 is an example weighting process for use with the system of FIGS. 1A-1B.

FIG. 9 is a flow chart of an example scheduling process for use with the system of FIGS. 1A-1B.

FIG. 10 is a flow chart of an example weighted scheduling process for use with the system of FIGS. 1A-1B.

The illustrated implementations are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

The systems described herein may include a scheduling system to provide patient-centric scheduling that may revolutionize medical appointment and resource scheduling. For example, patient-centric scheduling may increase patient satisfaction, patient compliance (e.g., fewer missed appointments and adherence to required fluid volume removal in a given week), and potential to avoid unnecessary hospitalizations and improve patient clinical outcomes. The systems and methods described herein may enable patient-centric scheduling for medical appointments and other services including, but not limited to clinical services, surgical services, or other treatment-based service. In addition, the systems and methods described herein may provide an advantage of lessening morbidity and/or mortality risks as compared to conventional scheduling systems and/or conventional scheduling techniques.

In some embodiments, the systems and methods described herein may be used to schedule people, equipment, and/or other resources for dialysis treatments in an optimized manner for the patient. For example, patients may use the systems to schedule multiple treatment dates according to a user-preferred schedule that enables schedule autonomy while ensuring equipment, staff, and/or other resources are available.

In some embodiments, the systems and methods described herein may be used to schedule people, equipment, and/or other resources for chemotherapy treatments. For example, patients can use the systems to schedule infusion sessions, infusion pumps, or other equipment or resources in a center that typically treats multiple patients.

In some embodiments, the systems and methods described herein may be used to schedule people, equipment, and/or other resources for radiotherapy sessions. For example, patients can use the systems to schedule radiation therapy on specific machines to deliver a prescribed dosage—such as with different types of external beam radiation and image-guided radiation therapy to ensure a cohesive treatment. Similarly, the patient may use the systems to schedule internal, infusion radiation that involves equipment (e.g., intravenous pump, etc.).

In some embodiments, the systems and methods described herein may be used to schedule people, equipment, and/or other resources for imaging procedures with CT, MR, X-ray, and/or ultrasound technologies. For example, patients may use the systems to schedule imaging procedures to coincide before or after another scheduled treatment or may schedule such imaging procedures according to a location at which the other scheduled treatment is scheduled to occur.

In some embodiments, the systems and methods described herein may be used to schedule people, equipment, and/or other resources for infection treatment infusions with intravenous pumps. In some embodiments, the systems and methods described herein may be used to schedule people, equipment, and/or other resources for hyperbaric oxygen therapy in chambers. In some embodiments, the systems and methods described herein may be used to schedule people, equipment, and/or other resources for physical therapy sessions on specific equipment and/or at specific locations. In some embodiments, the systems and methods described herein may be used to schedule people, equipment, and/or other resources for dental procedures utilizing specific chair capacity, equipment, or facility location. In some embodiments, the systems and methods described herein may be used to schedule people, equipment, and/or other resources for HIV infusions, in-hospital diabetes treatment, and/or other disease treatment.

In some embodiments, the systems and methods described herein may use particular algorithms and/or machine learning (ML) algorithms (e.g., neural networks, ML models, training data, etc.) to optimize dialysis scheduling for each user (e.g., patient) while ensuring safety and regulatory parameters and staffing needs are met. For example, the systems and methods described herein may include the use of algorithms and/or ML models to enable the patient to conveniently schedule and reschedule dialysis appointments in a way that optimizes scheduling according to equipment availability, staff (e.g., technician, physicians, etc.) availability, and/or patient dialysis prescriptions or needs.

In some embodiments, the scheduling system may be operated in a mode to bias the output of scheduled events according to a selected entity. In some embodiments, the selected entity may include the patient and the output may include appointments selected to provide convenience and timing based on health needs of the patient. In some embodiments, the selected entity may include the staff of a facility and the output may include appointments selected to ensure convenience for the staff according to staff schedules, staff profiles, or other staff input available to the scheduling system. In some embodiments, the selected entity may include the equipment of a facility and the output may include appointments selected to ensure equipment availability and/or backup equipment availability as well as any staff based schedule that may be associated with particular equipment. For example, particular staff members may be deemed as experts for particular equipment. The scheduling system may ensure that both specific equipment and specific staff members are available before generating a suggested appointment (and/or appointment schedule) for a patient. In this way, the scheduling system may facilitate requests from multiple parties and use equipment configuration and/or availability data to ensure that both staff and patients are satisfied with the scheduling of each dialysis appointment.

In addition, when patient data is available, the scheduling system can ensure that each patient's health is considered when generating dialysis schedules for many patients across any number of facility locations. In some embodiments, the systems described herein may further optimize scheduling for multiple dialysis centers in a region (including the multiple machines and technicians at each center), drive times for remote patients, weather delay risks, and lifestyle choices in between dialysis sessions, etc.

In some embodiments, the scheduling system may be a two layer system that includes a patient schedule and a staffing schedule. The patient schedule may be matched to particular equipment schedules and then further matched with particular staff based on the staffing schedule. For example, the scheduling system may incorporate, for each patient, a patient profile which would determine specific time requirements for scheduling equipment such as a dialysis machine and/or additional equipment that may include additional set up time. In one non-limiting example, the scheduling system may use a patient profile to determine that a particular patient utilizes a Hoyer lift and accordingly, the system may arrange additional appointment time to account for staff schedules and extended equipment usage time. Similarly, the scheduling system may incorporate a staff employee profile, which may include certain credentials that allow specific patient ratios of care, seniority details, and/or an indication of ability to work alone on a shift.

In some embodiments, the scheduling system is a three layer system that includes the patient schedule, the staffing schedule, and a layer that also considers a physician schedule in situations where a physician's appointment and a dialysis appointment are being requested to the scheduling system. This third layer may provide an efficiency for both patients and physicians by providing multiple services in a single appointment.

In some embodiments, the scheduling system described herein provides an advantages of accurate automation of scheduling by eliminating double booking conflicts across calendars, reducing human error from manual data entry, and providing audit trails and version histories for appointment scheduling. In addition, the scheduling system may increase efficiency of scheduling because such systems may reduce times for finding openings across multiple calendars and schedules without the use of administrative staff such that the staff can focus on higher value tasks. The scheduling system may increase efficiency of scheduling because the system allows self-service rescheduling of appointments within defined parameters. In addition, schedules for patients, staff, physicians, and equipment may be optimized and real time messaging can ensure that schedules remain clear and concise.

The systems and methods described herein provide an advantage of increasing attendance rates for booked appointments and reducing late cancellations due to double booking because the scheduling system described herein may share and gather availability and/or preference data within a single system. For example, the systems and methods described herein may ensure that a patient can use an easily accessible app to select a series of available appointments to undergo dialysis sessions according to a physician's orders. For example, the scheduling system described herein may assess a patient schedule, an equipment schedule, a staff schedule, and/or a physician schedule and provide available appointments (or appointment series). The patient may accept such appointments/series and/or further request additional appointments, information, statuses, or other data available to the scheduling system. In some embodiments, a patient may submit particular time slots and the systems described herein may algorithmically determine a location (or multiple locations) in which the dialysis appointment(s) can be performed. In particular, the system may perform a number of comparisons, synchronizations, and/or negotiating steps to determine a schedule of appointments that may be conveniently scheduled according to patient requested blocks of time while still meeting the patient health needs, the staff schedules, the physician schedules, and the equipment availability schedules, as will be described in further detail herein.

The system 102 functions to optimize scheduling for patients, equipment, clinicians, and/or facilities. In some embodiments, the system 102 functions to optimize scheduling for dialysis machine usage. In some embodiments, the system 102 functions to optimize scheduling for multiple pieces of medical equipment. The system 102 is used for medical device scheduling and patient scheduling but can additionally or alternatively be used for any suitable applications, clinical or otherwise. The system 102 can be configured and/or adapted to function for any suitable scheduling purpose.

The systems and methods described herein solve a technical problem of determining suitable scheduling for a patient, equipment, and a clinician based on patient availability and/or preferences, equipment availability, and clinician availability and/or preferences. In particular, the technical problem sought to be solved by the present disclosure is to provide scheduling that optimizes use of equipment and clinician time while taking into account patient requests and/or preferences.

The system 102 can be used to optimize scheduling based on reward-based algorithms, which provides a technical effect of enabling selection of desired appointment scheduling to maximize convenience for a patient, usage of equipment, and to maximize clinician time.

Conventional systems and/or methods may utilize a clinician schedule when determining patient appointments. A potential drawback with such conventional solutions may not account for patient preferences, machine outages or availability, schedule changes, or the like. Thus, the systems and/or methods described herein may provide an improvement over conventional solutions by using any or all of the primary strategies of existing conventional systems as a reward mechanism that may select from (and/or modify) one or more existing scheduling approaches based on a system determined outcome or may instead select or generate a new scheduling approach based on analysis of prior approaches and scheduling options generated by system 102. The scheduling strategy may be selected on a per-patient basis from a variety of provided approaches, for example, in addition to being able to introduce new approaches into the existing system 102.

In addition to being flexible in the ability to schedule patients for Dialysis or other medical or non-medical appointments, the system 102 may also be extensible and able to support multiple types and strategies of appointments concurrently. If the system 102 is orchestrating appointments for multiple services within the same system, the system 102 may make more competent suggestions as a result of having more overall data available.

In a non-limiting example, the scheduling system 102 described herein may utilize rules, calendars, patient data, and/or ranking engine to determine how to schedule patients, staff, equipment, and/or physicians such that a patient can provide input on (or otherwise influence) the scheduling tasks. For example, the scheduling system 102 may generate appointment suggestions by first obtaining calendar data (or other schedule-based data) for any number of patients, staff, physicians, and/or equipment. The system 102 may then normalize the calendars to a singular format (e.g., convert differences in date/time formats, etc.). The system 102 may assess the normalized data to identify busy (e.g., unavailable) times for each assessed calendar by parsing the calendar data to identify periods of time marked as busy or otherwise blocked. The system 102 may aggregate the busy times for all calendars by combining busy times across the calendars into a master list, for example. The system 102 may identify open (e.g., available) time slots within the aggregated data according to rules, calendars, patient data, ranking data, or the like. The system 102 may then filter the available time slots according to particular constraints (e.g., minimum time available, patient constraint, physician constraint, staff constraint, equipment constraint, etc.). Upon filtering the available time slots, the system 102 may determine (and/or rank) one or more time slots that may be presented to the patient for selection. In response to receiving a patient input selecting one of the one or more time slots, the system 102 may generate appointment(s) according to the patient-selected time slot(s).

In some embodiments, the system 102 may further rank the available time slots according to the constraints and/or another user-based or system-based input. For example, the system 102 may rank available time slots before presenting such time slots. In some embodiments, the ranking may be applied according to a longest duration available to prioritize time slots with the longest continuous availability across all calendars. In some embodiments, the ranking may be applied according to time slots in the middle of the day (e.g. 10 AM-4 PM). In some embodiments, the ranking may be applied according to a surrounding context, for example, to boost slots that are surrounded by longer available blocks of time to allow for logistical changes to equipment and/or buffer time between patients. In some embodiments, the ranking may be applied according to patient preferences, for example, to allow each patient to specify preferred meeting times or windows of time. In some embodiments, the ranking may be applied according to a preferred day of the week to account for typical schedules by staff, physicians, and/or patients when ranking across different days. Monday morning may be preferable to Friday afternoon for example. In some embodiments, the ranking may be applied according to a patient or facility location and/or a patient commute time. For example, if locations are provided in calendars, the system 102 maybe give preference to time slots (and locations) in which patients would have shorter commutes to a particular location. In some embodiments, the calendars and preferences described herein may be received as input to the system 102 before time slots are determined. In some embodiments, the calendars and preferences described herein may be received as input to the system 102 after the time slots are determined and provided to the patient. For example, the patient may choose to upload a calendar or enter data pertaining to preferences after determining the system-provided time slots are not conducive to the schedule of the patient. In response, the system 102 may reperform the task of determining one or more time slots in which to schedule the patient based on the received patient calendar(s) and/or input.

When equipment (e.g., medical equipment, such as a dialysis machine) is to be included in the scheduling, the system 102 may integrate with an equipment reservation system via an API, exported data, web scrape, or other data accessing process. To account for equipment availability and scheduling, the system 102 may utilize a number of additional contextual ranking and/or contextual constraints. For example, the system 102 may assess reserved equipment times, maintenance schedules, and/or setup/takedown time to ensure that the equipment is available for any generated appointment suggestions. In addition, staffing availability for operating such equipment may also be considered when generating appointment suggestions for patients.

The systems and methods described herein may provide patients with an option to self-select time slots (e.g., appointments) for completing a prescribed dialysis regimen. Such a regimen (or prescription) may be prescribed by a physician. The regimen may include dialysis parameters that dictate minimum and maximum weekly dialysis requirements for a particular patient. Accordingly, patient self-selected time slots will be tailored to the parameters set by the prescription. The appointments provided by system 102 may be presented for patient selection on a computing device such as a wearable device, a smartwatch, a mobile device, a tablet device, a laptop device, or the like. Patients can input selections for an upcoming week and beyond—out into the future on a weekly basis for several months. In some embodiments, the patient may select appointments for about 6 weeks to about 8 weeks into the future such that dialysis may be performed regularly over a prescribed amount of time.

The systems and methods described herein provide an advantage of ensuring that clinicians (e.g., staff, physicians, etc.) may prescribe a dialysis regimen that prompts the patient to self-select an appropriate amount of dialysis sessions (e.g., appointments) and timing according to the prescribed regimen. The prescribed regimen may also set boundaries for a frequency of dialysis, a minimum and maximum length of time between sessions, a minimum and maximum length of sessions, and the like. In some embodiments, the staff and/or physicians may generate a patient profile for a patient that describes patient acuity, co-morbidities, and/or any special requirements for facilitating treatments, such as the need for a Hoyer lift, isolation, or an increased nursing ratio or staff ratio, etc.

The systems and methods described herein may enable staff (e.g., nurses, technicians, etc.) to provide operating windows of time for the available capacity of the dialysis equipment. This includes indicating a ramp up and ramp down time used to prepare the dialysis machine between patients. The staff may review patient profiles to ensure adequate parameters are in place for dialysis configuration time on a patient-specific basis.

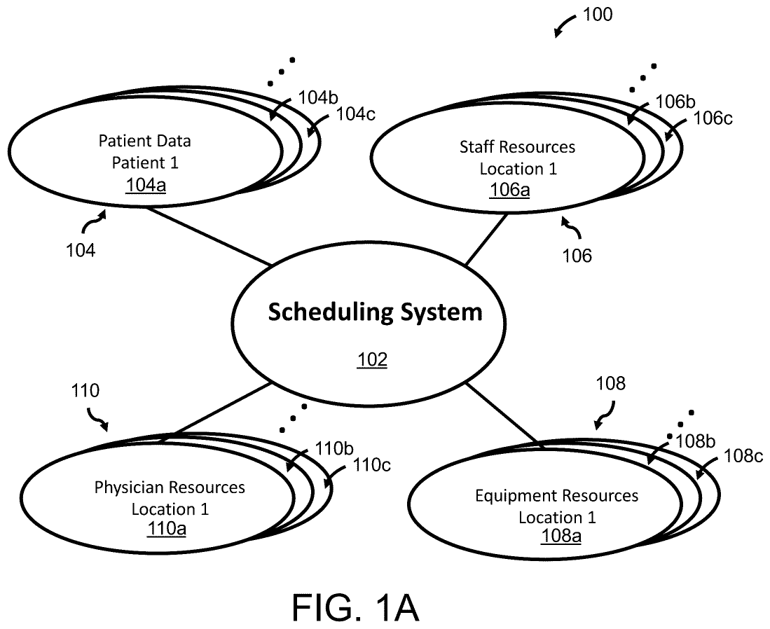

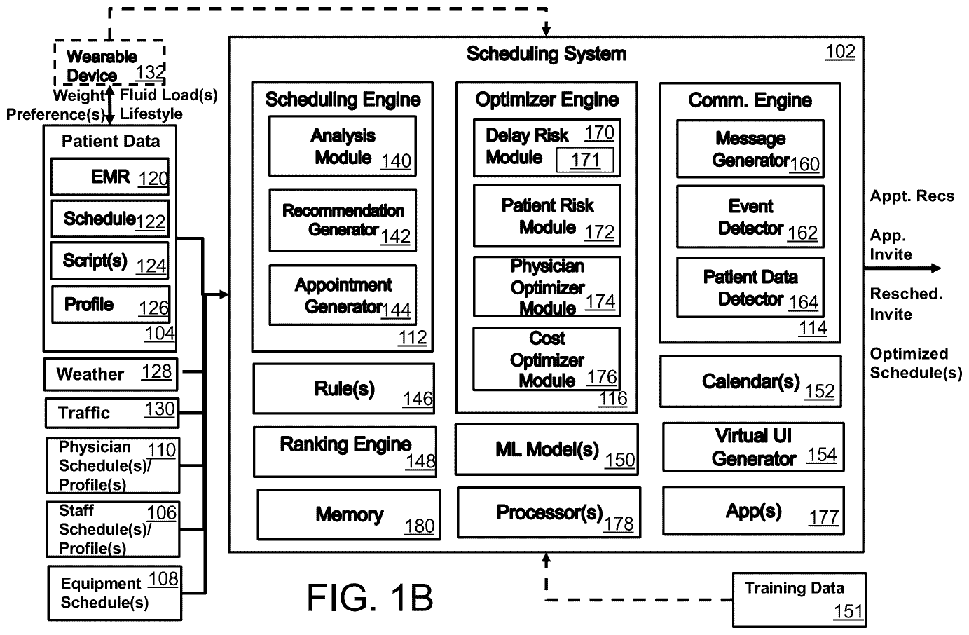

FIG. 1A illustrates an example high level system 100 for generating dynamically optimized schedules for dialysis management. The system 100 may include a dialysis scheduling system 102 with access to a number of data inputs that may take the form of patient data 104, application programming interface (API) input, requested input, schedule and calendar input, equipment planning data and maintenance, staff data 106, equipment data 108, physician data 110, training data 151, and/or system variables. For example, the system 100 may include, request, or receive any number of inputs from patient data 104, staff resources 106, equipment resources 108, and/or physician resources 110. In particular, the scheduling system 102 may obtain or request input from any number of locations that schedule patients, house equipment, and employ staff and/or physicians. Each location may be associated with multiple schedules that may be used to generate a master schedule for the location (or group of locations). For example, the patient data 104 may include a number of patients (e.g., Patient 1104a, Patient 2 104b, Patient 3 104c, etc.). Similarly, the staff resources 106 may include staff and staff schedules associated with Location 1 (e.g., staff resources 106a), staff schedules associated with Location 2 (e.g., staff resources 106b), staff schedules associated with Location 3 (e.g., staff resources 106c), etc. Equipment resources 108 may include equipment schedules and/or usage data for equipment resources at Location 1 (e.g., equipment resources 108a), equipment schedules and/or usage data for equipment resources at Location 2 (e.g., equipment resources 108b), equipment schedules and/or usage data for equipment resources at Location 3 (e.g., equipment resources 108c), etc. Physician resources 110 may include physician schedules and associated data for physician resources at Location 1 (e.g., physician resources 110a), physician schedules and associated data for physician resources at Location 2 (e.g., physician resources 110b), physician schedules and associated data for physician resources at Location 3 (e.g., physician resources 110c), etc.

In operation, the system 100 may receive inputs such as a dialysis prescription including a frequency and duration of dialysis procedures for one or more patients, availability of dialysis equipment, and availability of one or more staff (e.g., technician, nurse, etc.) to operate the dialysis equipment. With such information, the system 100 may generate possible scheduling suggestions for at least one patient to receive dialysis according to the prescription for the at least one patient. For example, the system 100 may utilize scheduling system 102 to determine one or more dates and time periods within the one or more dates in which the patient could receive dialysis based on the frequency and the duration of dialysis (e.g., the prescription). The determined one or more dates and the time periods are generated taking into account the inputs such that the generated suggested appointment(s) are aligned with the availability of the one or more dialysis machines and the availability of the one or more staff.

In general, the scheduling system 102 may generate appointment suggestions (e.g., indications, meeting requests, etc.) for transmission to a patient. The appointments may be determined by the system 102 as a way to automatically schedule equipment, patients, staff, and/or physicians, and may do so even when equipment, patients, staff, physicians, and multiple locations exponentially increase in number.

In operation, the system 102 may generate appointment suggestions by first obtaining calendar data (e.g., planning data, profile data, preference data, etc.) for any number of patients, staff, physicians, and/or equipment. The system 102 may also obtain equipment reservation schedules (e.g., from a reservation system or equipment tracking database or the like). The system 102 may then normalize data formats to consistent date and/or time fields to begin comparisons amongst the normalized data to identify busy times for each patient, staff, and equipment reservations. The system 102 may aggregate the busy times into a master schedule and identify openings in the schedule for a number of combinations of patient-staff-equipment combinations according to one or more constraints including, but not limited to profile preferences, time availability, prescription or health needs of each patient, rules for a particular location or equipment, buffer time to setup or take down equipment, etc. Upon finding valid days and times in which a particular patient can be scheduled according to their prescription and with appropriate staff, equipment, and/or physician, the system 102 may rank available schedule openings (e.g., appointment suggestion) according to any number of other constraints or preferences as described in further detail herein. The scheduling system 102 may generate and transmit an indication of the ranked available appointment suggestions to the patient for review and selection. Upon receiving a selection from the patient, the system 102 may schedule the appointment(s) and schedule any staff, physician, or equipment reservation(s).

FIG. 1B illustrates a detailed view of the example system 100 for generating dynamically optimized schedules for dialysis management. As shown, the scheduling system 102 includes a scheduling engine 112, a communication engine 114, and an optimizer engine 116. The scheduling engine 112 may interface with both the optimizer engine 116 and the communication engine 114 to generate appointment suggestions for patients and to schedule appointments for medical sessions, such as dialysis treatments.

The scheduling system 102 may receive or retrieve (with permission) electronic medical record (EMR) data 120, patient schedule data 122, patient prescription data 124, and/or patient profile data 126 as a basis in which to generate additional suggested appointments and messages pertaining to existing appointments and patient behavior. In addition, the scheduling system 102 may receive or retrieve weather data 128 and traffic data 130 to be used for determining the timing in which to trigger reminder notifications and/or departure notifications for appointments. In some embodiments, the weather data 128 and/or traffic data 130 may further be used to determine a likelihood of patient tardiness or absence. The scheduling system 102 may also receive or retrieve physician schedules and/or profiles 110, staff schedules and/or profiles 106, and equipment schedules 108.

In some embodiments, the scheduling system 102 may further receive or retrieve patient weight changes over time, patient preferences, patient fluid loads, patient lifestyle metrics, or the like, from an optional wearable device 132. In some embodiments, the optional wearable device 132 is a smartwatch. In some embodiments, the optional wearable device 132 is a patch or adhesive-based sensor. In some embodiments, the wearable device 132 is an implanted device. In some embodiments, the optional wearable device 132 is a computing device in which the patient may enter data for system 102 to analyze.

In some embodiments, the scheduling system 102 may further receive or retrieve patient weight changes over time, patient preferences, patient fluid loads, patient lifestyle metrics, or the like, from a communicatively coupled electronic device (e.g., having received one or more inputs related to the patient). In some embodiments, the electronic device is a mobile phone, laptop, desktop computer, netbook, tablet, or the like.

Referring back to the scheduling engine 112, a number of programmed elements may be used to analyze input and generate appointment suggestions. For example, the scheduling engine 112 includes an analysis module 140, a recommendation generator 142, and an appointment generator 144. The analysis module 140 may utilize two or more of patient data 104, physician schedules/profiles 110, staff schedules/profiles 106, equipment schedules 108, weather data 128, and/or traffic data 130 to determine overlaps in availability of resources available to system 102 and assess when particular messaging is to be transmitted from system 102. For example, the analysis module 140 may access data including reservation data and/or calendar data for each location and within each location may assess equipment schedules 108, staff schedules/profiles 106, and patient data 104, for example. The data may be integrated into a single system or assessed on an ad-hoc basis. The analysis module 140 may assess the data to determine whether particular location logistics should be accounted for when scheduling particular patients based on a distance from the patient location (e.g., tracked location, home address, work address, etc.). The analysis module 140 may assess the data to coordinate overhead scheduling of a particular location by finding openings that work across more calendars and equipment. In some embodiments, the module 140 may determine that a piece of equipment may be used with more patients by relocating or transporting the equipment on a temporary or permanent basis. The analysis module 140 may use rules 146, for example that may include location-specific rules and/or equipment-specific rules, each of which may elucidate administrative rules, operating and permission rules, or the like. In some embodiments, the rules 146 represent a domain specific language used to define and express rules (e.g., similar to LLM prompts).

In some embodiments, the analysis module 140 may also use ranking engine 148 to rank appointments and/or appointment suggestions. The ranking engine 148 may utilize ranking criteria to weight scheduling criteria and/or appointment suggestions. The ranking criteria may be based on one or more inputs (e.g., patient data 104, staff data 106, equipment data 108, physician data 110, training data 151, and/or system variables).

In some embodiments, the analysis module 140 may further use one or more machine learning (ML) models 150 to perform schedule planning tasks and schedule generating tasks. In some embodiments, the ranking engine 148 may directly access the ML models 150 to perform ranking tasks. For example, the analysis module 140 may access one or more ML models 150 to process patient and appointment data from any number of input sources and may provide normalized outputs for future processes of identifying appointments to be scheduled for a user. The recommendation generator 142 may use output from the analysis module 140 and apply the Epsilon-Greedy algorithm described herein and/or the Monte Carlo simulations to identify optimized appointment times for a user.

The ML models 150 (e.g., neural networks, ML models, training data, etc.) may be used to generate and/or optimize dialysis scheduling for each user (e.g., patient) while ensuring safety and regulatory parameters and staffing needs are met. For example, the systems and methods described herein may include the use of algorithms and/or ML models 150 to enable the patient to conveniently schedule and reschedule dialysis appointments in a way that optimizes scheduling according to equipment availability, staff (e.g., technician, physicians, etc.) availability, and/or patient dialysis prescriptions or needs.

In general, the ML models 150 may include neural networks that may be trained to perform constraint optimization modeling, regression modeling, similarity modeling, queue modeling, or other modeling technique to assess patients, staff, and physicians in combination and to further generate possible appointment suggestions that fit the performed modeling. In some embodiments, the ML models 150 may utilize reinforcement learning models to learn scheduling policies over time, for example. In some embodiments, the ML models 150 may utilize graph neural networks to represent physicians, clinic locations, patients, staff, and available equipment. The graph neural network (GNN) may model interactions and connections and optimize assignment of patients to staff and/or physicians. In some embodiments, the ML models 150 may utilize queuing models to predict a number of patients arriving over time and wait times or backlogs occurring at a clinic location, for example. Such models may schedule based on the queue dynamics assessed using the queuing models. In some embodiments, the ML models 150 may utilize regression models to predict appointment durations, no show patients, and to forecast appointment time lengths according to staff or procedure. In some embodiments, the ML models 150 may include or represent large language models (LLMs) that interpret prompts and generate scheduling recommendations, as described elsewhere herein. In some embodiments, the ML models 150 may include or represent a library of ML models, which may be applied to solving various types of scheduling concerns.

In some embodiments, the ML models 150 may be trained using training data 151. The training data 151 may include, but is not limited to logs of historical schedule and/or appointment records, demand for appointment data, historical waitlisted metrics for patients awaiting appointments, historical availability records for staff, physicians, and equipment, historical maintenance records for equipment, etc. In some embodiments, training data 151 may be collected by working with clinic staff at one or more locations to obtain structured data, patient demographics, appointment types, provider calendars/availability, cancellation data, etc. Such data may be anonymized to remove identifiable information before being used as training data. In one non-limiting example, the ML models 150 may be trained on clinic data for any number of locations to predict optimal appointment slots for dialysis. Specialty-specific appointment patterns and/or durations can be incorporated. The performance of the trained ML models 150 may be validated before deployment.

In some embodiments, historical appointment data (e.g., including timeliness of the appointment) may enable the system 102 to provide insightful recommendations upon the first appointment using it for each patient. Each subsequent appointment may inform the system 102 to enable the system 102 to learn about appointment/patient behavior over time. Without such data, the system 102 may use the training data 151 to determine appointment recommendations. Input from one or more wireless devices 132, patient data 104, weather 128, traffic 130, or the like can be used to inform the recommendations generated by system 102. For example, when generating recommendations for dialysis scheduling, the system 102 may consider blood records and treatment data in addition to schedule events for staff/clinicians and patients.

The ML models 150 may generate algorithms that use machine learning techniques to generate smart schedules that account for patient preferences, staff preferences, physician preferences, and equipment efficiencies. The ML models 150 may determine appointment suggestions for a patient based on any number of constraints or inputs, as described in detail herein. The ML models 150 may work with scheduling system 102 to generate UI content for presenting the appointment suggestions to a patient (e.g., user) and to receive input from the user. The ML models 150 may use such input to generate additional appointment suggestions, schedule a selected appointment, trigger messaging, trigger requests, and/or to provide additional information to patients, staff, and/or physicians.

In some embodiments, the system 102 may utilize model free learning (i.e., without ML models 150) and such learning may derive data from real world scheduling over time. Model free learning may be executed by processor 178 and memory 180 in combination with scheduling engine 112, communication engine 114, and/or optimizer engine 116. For example, the system 102 may utilize model a model-free reinforcement learning algorithm such as Q-learning that employs temporal differences where predictions are evaluated after each step in the learning algorithm.

In some embodiments, the system 102 may utilize an Epsilon-Greedy Q-learning algorithm to construct a flexible, model free system for optimizing schedules. Using the Epsilon-Greedy Q-Learning algorithm may ensure that the system 102 balances itself between exploration and exploitation. For example, the Epsilon-Greedy Q-Learning algorithm may select from one of a fixed pool of potential choices and may learn which option produces a highest number of rewards over time. The epsilon value may control the ratio of exploration to exploitation, and it can be dynamically modified each iteration to adjust the behavior of the system 102. The closer a value is to zero, the more the system is set to Exploit versus Explore. By tracking the win rate (e.g., successful scheduling event) versus loss rate (e.g., unsuccessful or erroneous scheduling event) for each of the strategies, the system may adjust the epsilon value to become more or less exploratory as a result of the success or failure of our scheduling strategies.

The scheduling strategies may be represented by software algorithms that conform to the same interface. This allows individual strategies to prioritize different pieces of data in an approach to generating a successful result. An example may include an instance of the strategy for every appointment block throughout the day. Each patient can be treated as a new element in the system 102 and each strategy may include an appointment day and time. Initially, the system 102 may offer the patient all the available time slots, and the time slots that are selected may be considered a win. Until the system 102 executes enough iterations of the algorithm to tip into exploitation mode, the system 102 may continue suggesting available time windows that have a high likelihood of success from past scheduling events.

In this example, patients can be weighted in the algorithm, as well as weighted according to a patient cancellation rate. These weights can be used to estimate cancellation rates on a given day based on the traits of the patients scheduled that day. In some embodiments, the system 102 may optimize the schedule to not schedule multiple patients in a given day that are prone to cancellations. Intentionally scheduling a balanced ratio of patients likely to cancel versus patients that are historically reliable could serve as a means of reducing pressure from staff while making an honest attempt at giving patients appointment opportunities.

In operation, the system 102 may present a patient appointment as a choice to the Epsilon-Greedy algorithm. A variety of weighted factors may be used to produce a reward to the algorithm, such as punctuality, equipment availability, and/or dialysis outcomes, for example. Appointments can be derived as a function of dialysis machines (or other equipment) multiplied by a number of time windows per day in which such machine are usable, as well as staff availability. The system 102 may function to balance staff, equipment, and patients to optimize for an ideal utilization rate of equipment, resources, and the like. For example, a maximum efficiency rate may be defined as an ideal utilization rate. In the example of dialysis equipment, the system 102 may define and 80% maximum efficiency as the ideal utilization rate to allow for emergencies and unforeseen disruptions that may take such equipment out of service.

In some embodiments, the scheduling system 102 may carry out Monte-Carlo simulations on historical appointment data to provide recommended appointment schedules when a patient is initially introduced to the system 102, for example. Monte-Carlo simulations may be used in an ongoing basis to derive next-appointment recommendations and/or to forecast anticipated risk for each choice entered into system 102.

In some embodiments, the system 102 may incorporate an unlimited number of potential scheduling strategies, such as a next day policy, a next available day policy, a shortest queue policy, and any others that may be identified over time. These alternative policy types can be presented as another type of abstract machine for the Epsilon-Greedy algorithm to explore. Additional policy types may be used instead of, or in addition to other types of strategies, among the same or different patients within the system.

In some embodiments, the recommendation generator 142 may obtain data from the analysis module 140, ranking engine 148, ML models 150, and/or calendars 152 to generate one or more recommended appointments for a particular patient. In some embodiments, the ML models 150 may be configured to perform patient and schedule analysis with analysis module 140 while utilizing pattern classification to match patient requests to system 102 availabilities. In such an example, the ML models 150 may be configured to use output from the analysis module 140 to generate recommendations for appointments in which to suggest to patients. Such recommendations may be generated by recommendation generator 142, which may function with the ML models 150 to generate the recommendations. In some embodiments, the recommendation generator 142 may instead utilize historical records and other input to generate recommendations for a patient without utilizing ML models 150.

In general, a recommendation generated by recommendation generator 142 may be a desired scheduling recommendation or an undesired scheduling recommendation. The system 102 may weight such recommendations according to categories such as user requests, punctuality of users or clinicians, medical outcomes, personality conflicts (e.g., staff vs. patient, patient vs. patient), and/or acceptance ratio, etc. The weighting may be based on any number of categories and such categories can be incorporated into a scheduling strategy for a particular patient, clinician, or location.

In a non-limiting example, a punctuality metric may be defined by the system 102 as being a metric of a patient check in time with respect to a scheduled time of an appointment. If a patient is chronically a number of minutes late for an appointment with a first piece of equipment, the system 102 may use the punctuality metric associated with the patient record, for example, to recommend a later appointment at an updated time slot with a second piece of equipment to ensure that other appointments on the first piece of equipment are not delayed. If the patient is then consistently on time, the updated time slot may increase in weight over time indicating that the later time slot is a preferred or more convenient time slot. Conversely, if the patient continues to be chronically late for the updated time slot, the recommendation indicating the updated time slot may lose weight and other time slots may instead be suggested.

In one non-limiting example, a personality conflict metric may be accounted for in recommendations provided by system 102. The personality conflict metric may represent a patient-indicated or clinician-indicated desire for scheduling appointments with particular patients/clinicians/locations (or lack of desire). In general, personality conflict metrics may be weighted using a zero (e.g., do not suggest appointment with clinician, location, etc.) or a one (feel free to suggest an appointment with clinician, location, etc.). Such metrics and weightings may allow the system 102 to optimize against scheduling two parties with conflict during the same time period, and/or may minimize the overlap to the extent possible.

In one non-limiting example, an acceptance ratio metric may be accounted for in recommendations provided by system 102. The acceptance ratio metric may represent a number of times in which the scheduling staff has accepted or rejected a given time slot. Each acceptance may increase the weight, while each active rejection may decrease the weight.

In one non-limiting example, a medical outcome metric may be accounted for in recommendations provided by system 102. The medical outcome metric may represent previous medical outcomes exhibited by the patient and/or the clinician. Medical outcome metrics are generally dependent upon the type of scheduling being conducted. When using medical outcome metrics as a basis for recommending appointment times/days,, scheduling data as well as EMR data 120 may be utilized by the algorithm in determining an ideal next appointment for dialysis patients, for example.

Other metrics are of course possible, as one skilled in the art will appreciate. In addition, any combination of the metrics may be used to generate a recommendation for an appointment.

In some embodiments, references to the system 102 may refer to components of FIG. 1B functioning to optimize scheduling, equipment usage, clinician time, or the like. In some embodiments, the EMR data 120 may be used to generate various weighted parameters on a patient record within the system 102. Similarly, schedule data 122 may represent historic schedule data used to establish baseline starting points when a patient is new to the system 102. Script data 124 may include descriptions of events relating to care to be followed on a repeating basis. Profile data 126 may include physiological attributes pertinent to diagnostic outcomes, which may represent a live status or state of a patient. The weather data 128 may represent an anticipated impact of future weather on upcoming scheduled events and may be used in forecast simulations. Traffic data 130 may represent regional traffic data ingested by an application programming interface to support the recommendation generator 142. Physician schedule data 110 may represent an availability of one or more physicians for an upcoming appointment. Staff schedule data 106 represents an availability of clinical staff. Equipment schedules 108 represent cleaning, maintenance, and/or other availability impacts for equipment used in appointments.

The appointment generator 144 may generate output including one or more indicators, appointments, user interface content, and calendar invites. For example, the appointment generator 144 may receive input from a patient that selected one or more of the schedule recommendations that recommendation generator 142 provided. Once the input is received, the appointment generator 144 may perform the scheduling of the selected appointment(s) for the patient. The appointment generator 144 may interface with available calendars 152 and virtual UI generator 154. For example, the appointment generator 144 may generate and provide information and UI screens (via virtual UI generator 154) to a patient to allow access to the scheduling system 102 and/or to interface virtually with one or more staff 106 or physicians 110. In some embodiments, patients may add additional clinical or personal appointments into a calendar 152 associated with system 102 to create a personalized scheduling availability. In some embodiments, the appointment generator 144 may represent user facing aspect of system 102 that may interact with the scheduling engine 112 to allow the user to take action on provided recommendations.

In some embodiments, the calendar 152 may include or have access to data from a food diary in which the patient may enter meals and other intake each day. The system 102 may utilize data in the food diary (with user permission) to track bodily levels or other metrics with respect to food, drink, and medicinal intake. For example, the system 102 may detect a high risk food and use such a detection as a trigger to send messaging or log data pertaining to the risk. In a non-limiting example, the user may enter a food with a high salt or high potassium load and the system 102 may trigger messaging to avoid additional potassium for the day or to schedule an appointment for particular treatment if some combination of the high potassium food and other detected event(s) indicates indicate an increased probability of having a health event or hospitalization event.

In some embodiments, the calendar 152 may include or have access to data from a blood pressure device or other smart sensing device to allow patient blood pressures to be considered by the system 102 as a basis in which to trigger messages and/or appointment scheduling or rescheduling.

The communication engine 114 includes a message generator 160, an event detector 162, and a patient data detector 164. The message generator 160 may generate messages for patients, staff, and physicians based on the data assessed by scheduling system 102. The messages can include reminders, alerts, instructions, directions, appointments, appointment suggestions, UI content, or the like. For example, the system 102 may be arranged to request that a patient schedule enough sessions to meet requirements of a specific prescription for dialysis and any corresponding fluid removal. In addition, the system 102 may further be arranged to provide specific default time blocks according to appointment types, equipment types, and/or staff availability to ensure that a default selection of dialysis times is provided with each suggested appointment time or block of time. This can also ensure that a clinic (i.e., staff) will have time to complete a specific treatment in the appointment. If the patient scheduled too few sessions to meet such prescription requirements, the message generator 160 may generate and transmit an alert message for the patient and a corresponding notification (e.g., message) for an associated physician and/or staff. The message generator 160 may also provide patients with reminder messages for upcoming appointments. In addition, the message generator 160 may send patients indicators or messages when an opening in the schedule is available to allow the patient an opportunity to be dialyzed at a different, potentially more convenient time than a previously scheduled appointment. The specific time slots offered to patients may be offered based on time requirements indicated in a patient profile 126 (e.g., patient acuity profile). Changes that occur in patient receipt of dialysis that deviate from their originally scheduled treatments, such as missed or late appointments, may trigger automatic updates to patient profiles and/or may trigger generation of messages for the patient to allow the patient to self-schedule additional dialysis sessions. Further, if a patient is identified as high or imminent risk of hospitalization as determined by system 102, for example, a message may be generated and transmitted to the patient and/or staff. The message may include dialysis times in which to schedule a dialysis session. For example, if system 102 determines that a patient is at imminent risk of hospitalization based on assessing patient compliance, historical trends, and/or clinical parameters such as dramatic weight changes, the system 102 may trigger generation and transmission of a message to mitigate the risk.

The message generator 160 may generate and transmit messages to a patient based on a record of past schedule compliance and/or recognition incentives for being on-time and consistently diligent in receiving dialysis treatments. Patients that are tardy or absent from scheduled appointments may receive messages and/or reports regarding the tardiness or absence. Such messages and/or reports may also be transmitted to staff and/or physicians associated with the particular patient.

The message generator 160 may generate and transmit messages to clinicians (e.g., staff, technicians, physicians, or the like). For example, the message generator 160 may provide report messages to clinicians. The report messages may detail patient compliance and notices of missed dialysis sessions. In some embodiments, the scheduling system 102 may automatically analyze the schedule and recommend specific days/times for the clinician to perform rounds on patients to be able to maximize the number of their patients to be seen according to a particular cadence for clinical objectives and/or reimbursement objectives. In this way, non-nephrologist clinicians who treat the patients have access to the dialysis schedule for their patients and can schedule visits—either in person or virtually—with the patient during their sessions and message generator 160 may generate messages accordingly.

The message generator 160 may further generate report messages for clinicians based on patient data 104 (e.g., EMR data 120, weight data, fluid load trend data, lifestyle data, impedance measurements, blood pressure, measurements, etc.) obtained from patient at-home scales and/or wearable devices 132, for example. Clinicians can then make short-term or long-term changes to the patient's prescription. In some embodiments, the patient data detector 164 may detect such data and work with the analysis module 140, ML models 150, and message generator 160 to generate messages associated with detected patient data.

In some embodiments, the system 102 may generate a hospitalization index (not shown) that assesses and reports a 30 day, 60 day, 90 day, and/or 120 day probability of hospitalization for any given patient based at least in part on historical trends, compliance, comorbidities, etc. The hospitalization index may continually refine as additional data is added or available for each patient.

In some embodiments, generating messages based on fluid load trend data may be triggered by detecting or receiving data indicating that a patient should begin treatments for restoring circulatory volume, clearing of ketones, correction of electrolyte imbalances, etc. The system 102 may detect events related to such indications as a basis in which to trigger messaging and schedule appointments. In this way, the system 102 may make a first contact of the patient to trigger scheduling or rescheduling of appointments based on the detected events rather than relying on the patient to make first contact of the medical clinic. In addition, scheduling or rescheduling based on imminent patient need can ensure that appointments are available for need-based (e.g., life threatening or hospitalization inducing) events.

The event detector 162 may function to generate and trigger reminder messages via the message generator 160. The reminder messages may be sent to patients, staff, physicians, equipment, or other device or entity that may be scheduled to encounter a patient at a particular time. In some embodiments, the event detector 162 may detect that a particular at-risk patient may be well served to be dialyzed earlier than a scheduled appointment. In such examples, the event detector 162 may work with scheduling engine 112 and/or optimizer engine 116 to find an earlier appointment in which to reschedule the patient.

In some embodiments, the event detector 162 and/or patient data detector 164 may be programmed to monitor and assess patients that have provided permission to be monitored and/or location tracked. The event detector 162 and/or patient data detector 164 may use such monitoring and/or tracking to aid in determining likelihoods of patient adherence to scheduled dialysis session attendance. The event detector 162 may also utilize weather data 128 and traffic data 130 to trigger messages indicating weather alerts or timing alerts for when the patient should depart to ensure on time appointment arrival.

In some embodiments, the scheduling system 102 may receive consent from patients to allow the system 102 to obtain demographic data such as home address, patient data, and/or medical record data via electronic access or other access linked to the scheduling system 102. For example, the patient may consent to allowing an at-home Internet-connected scale to be linked to the scheduling system 102 to trigger transmission of data to the staff and/or physicians to alert of any unusual patterns in weight and/or fluid load. In some embodiments, weight or other biological metric may be received at system 102 by connected systems including electronic medical record systems, other clinics, and/or communicatively coupled applications, wearables, or other device. Such alerts may facilitate a change in the prescribed dialysis in a specific week or time frame. This new prescription can trigger alerts to the patient to schedule additional (or fewer) dialysis sessions in the system 102. In addition, messages may be sent to a computing device of the patient as a reminder to capture periodic weight measurements. Compliance of at-home weighing are also reports that may be generated and provided by system 102. Similarly, wearable or implantable devices may send additional data to trigger alerts regarding fluid load, blood pressure, electrolytes, blood sugar, or the like.

In some embodiments, each staff member may provide a staff profile 106 and staff credentials into the scheduling system 102, which may influence a staff-to-patient ratio during any one shift. Alternatively, the scheduling system 102 may obtain such information from another source and not directly from the staff members. In some embodiments, the staff schedule 10 may include both a fixed and a flexible scheduling component. Supervising staff can set fixed parameters (i.e. black-out dates) in order to meet the needs of the dialysis units. With a certain probability, the system 102 can inform when shifts will be expected to be under-or over-staffed which will in turn generate a rescheduling opportunity for the staff. Available shifts/dates for staff scheduling, will also be guided by staff profiles 106. The staff profiles 106 may account for credentials, training, seniority, and/or other factors as determined by the supervisor which may influence the desired team composition for any given shift.

The staff may also receive report messages (e.g., from message generator 160) on patient schedule adherence and alerts for late or missed sessions. The system 102 can provide messages via generator 160 to inform nurses and technicians if certain shifts will have a projected patient census which could cause a re-scheduling opportunity for the staff. In some embodiments, the message generator 160 may provide report messages to staff regarding projected patient census from the self-scheduling along with staff assignments to project expected reimbursement revenue and operating expenses to calculate forecasted financial results. The report messages can also inform the ordering of dialysis supplies and indicate dialysis machine maintenance. In some embodiments, the message generator 160 may also provide report messages to staff based on the patient scheduling to suggest when lab samples should be drawn and batched for central processing. In some embodiments, the scheduling system 102 can predict, based on the projected patient schedules, when the optimum time for lab sampling should occur.

The optimizer engine 116 may function to optimize schedules according to one or more parameters including, but not limited to optimizing for equipment use, down time, and maintenance; optimizing for patient or physician schedules; optimizing costs of using equipment and staff or physician time; and/or optimizing for or reducing patient risks with respect to schedule-based failures or lack of availability of equipment, staff, and physicians. For example, the field of dialysis treatment is dominated by two conventional for-profit companies, who have utilized the historical precedent of treatment occurring three times per week (i.e., Monday, Wednesday, Friday or Tuesday, Thursday, Saturday) at in-center hemodialysis operations. The conventional focus is on optimizing the utilization of dialysis equipment and the staff to operate the equipment, in order to minimize the cost of treating patients and maximize the profit of the center from reimbursement income. These set schedules for patients also simplify the operation and management of the treatment center. This conventional approach ignores unique patient preferences, work schedules, child care schedules, and potential changes in diet/behavior. Inflexibility of the schedule may cause missed dialysis sessions, which in some cases can lead to unplanned hospitalization. Conventional systems schedule dialysis centers focused on optimizing the staffing levels and for financial planning, and do not factor in the patient clinical outcomes. The system 102 may be tuned to optimize in such a conventional fashion, but additionally system 102 may provide an ability to tune schedules according to any number of optimization parameters, as described in detail herein.

The optimizer engine 116 includes a delay risk module 170, a patient risk module 172, a physician optimizer module 174, and a cost optimizer module 176. The delay risk module 170 may receive input that indicates a particular risk or assessment of delay of a patient, staff, physician, or equipment. For example, the scheduling system 102 may receive an input indicating one or more of: a drive time for the patient (e.g., traffic data 130), a weather input of the location (e.g., weather data 128), and a delay risk metric 171. The delay risk metric 171 may be an input or alternatively may be calculated based on one or more of: traffic data 130, a historical appointment attendance for the patient (e.g., stored in memory 180, stored in an EMR, and/or patient data 104), a schedule of one or more additional dialysis machines at the location (e.g., stored in memory 180 and/or scheduling engine 112 and/or calendars 152), or a schedule of one or more additional staff at the location (e.g., stored in memory 180 and/or scheduling engine 112 and/or calendars 152). The input or calculated delay risk metric may be used by scheduling system 102 to automatically update one or more dates and time generated for a patient by system 102. For example, if the system 102, via delay risk module 170, detects that the patient may be behind schedule by about 15 minutes, the system may reschedule the appointment for a later time (e.g., 15 minutes delay or more) and may trigger rescheduling for the associated staff, physician, and/or equipment reserved for the original appointment.

The patient risk module 172 may utilize input such as appointment cancellations, appointment absences, patient data 104, data received from wearable device 132 (or other implanted device or computing device), or the like to determine whether a patient is at risk of hospitalization. For example, the patient risk module 172 may detect that a cancellation input (or absence) has been documented for a patient and in response, module 172 may generate and output a second appointment time on a date within the patient's prescription window of time (for completing dialysis treatments) such that the patient is not put at risk solely because the patient could not obtain a new appointment within the prescribed window of time for dialysis sessions. For example, the scheduling system 102 may attempt to reschedule the patient within the prescribed window of time in order to avoid having the patient experience increased potassium, toxin buildup, demineralization, pulmonary edema, or other cardiovascular or metabolic issue as a result of missing a dialysis appointment.

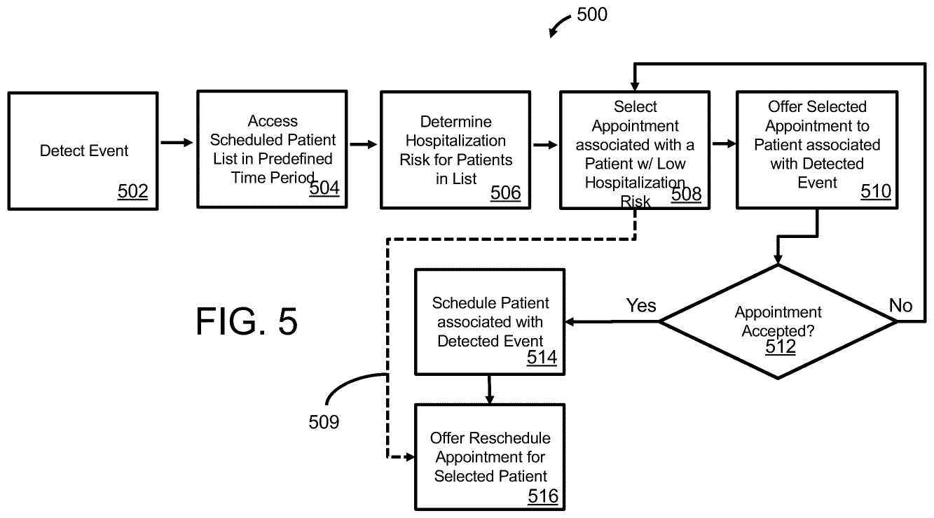

In one non-limiting example, the scheduling system 102 may determine that a patient that cancelled or missed an appointment may be at a higher hospitalization risk than another patient on the existing schedule. In response, the system 102 may attempt to reschedule the second (and lower hospitalization risk) patient in order to provide a time slot for the first patient that is at the higher hospitalization risk, as described in detail in process 500 of FIG. 5 below.

The physician optimizer module 174 may generate optimized scheduling for a set of physicians to ensure that each particular physician may attend to patients during a time in which the patient is undergoing dialysis at a facility and within a time slot in which the physician is scheduled. For example, a physician (e.g., clinician) may have a rounds schedule that includes performing an evaluation of each patient from one to four times per month. In order to optimize rounding for the physician, the physician optimizer module 174 may assess patient scheduled appointments as a basis in which to allow the physician to round on the greatest number of patients within particular time period. For example, the physician optimizer module 174 may automatically analyze the schedule and may recommend specific days/times for patients based on a probability of rounding on the greatest number of patients within the time period. In this way, the system 102 provides an efficient way of scheduling physicians by understanding the overall needs of patients seeking appointment blocks over several weeks or months.

The cost optimizer module 176 may generate optimized scheduling of patients, staff, physicians, and/or equipment based on one or more inputs (e.g., patient data 104, staff data 106, equipment data 108, physician data 110, training data 151, and/or system variables, etc.).

The scheduling system 102 further includes one or more applications 177 (Apps). The apps 177 may include any number of applications, APIs, or access points to scheduling system 102. For example, patients may access an app 177 to enter input (as described herein elsewhere) for tailoring a set of appointments to a particular schedule or location. In this way, the patient may be part of the scheduling process.

The scheduling system 102 includes processors 178 and memory 180. The processors 178 may include one or more processors that include one or more devices capable of executing instructions, such as instructions stored by the memory 180, to perform communications amongst wearable device 132, user devices (not shown), scheduling system 100, scheduling system 102 (e.g., scheduling engine 112, communication engine 114, optimizer engine 116, ML models 150, ranking engine 148, calendars 152, and/or virtual UI generator 154, and/or third-party integrations (not shown)).

The memory 180 can include one or more non-transitory computer-readable storage media. The memory 180 may store instructions and data that are usable in combination with processors 178 to execute the processes and/or algorithms described herein as well as to execute or interface with ML models 150, scheduling engine 112 tasks, communication engine 1114 tasks, and optimizer engine 116 tasks. The memory 180 may also function to store or have access to the ML models 150, scheduling engine 112, communication engine 114, and optimizer engine 116, and patient data 104, weather data 128, traffic data 130, physician schedules/profiles 110, staff schedules/profiles 106, and equipment schedules 108.

In some embodiments, the systems 100, 102 may further include or be communicatively coupled to input devices (not shown), output devices (not shown), and/or sensor interfaces (not shown). The input devices may interact with one or more processors 178, memory 180, and/or wearable device 132.

In some embodiments, the systems 100, 102 may further include or be communicatively coupled to output devices (not shown). The output devices may interact with one or more processors 178 and memory 180. In some embodiments, the output devices may include, for example, an external display for depicting user interfaces generated by virtual UI generator 154 and/or scheduling system 102.

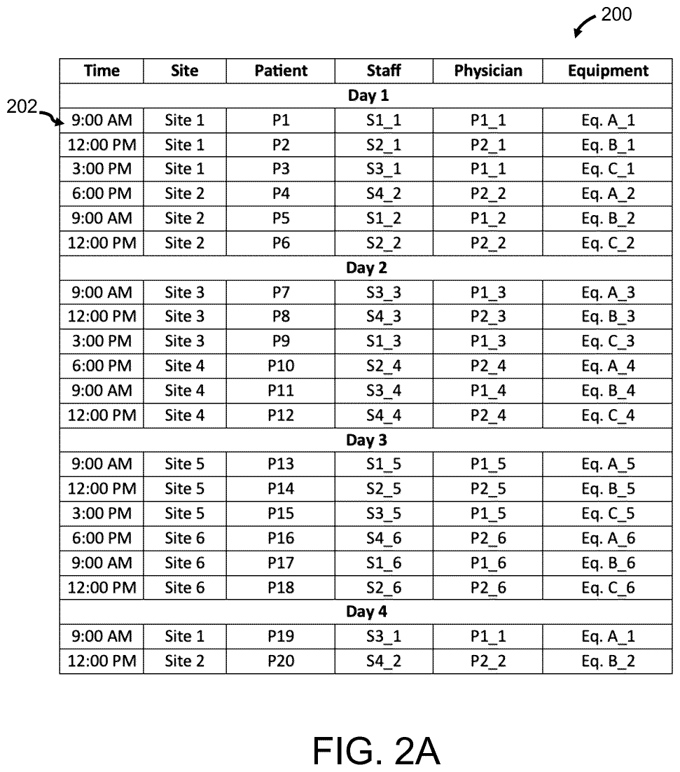

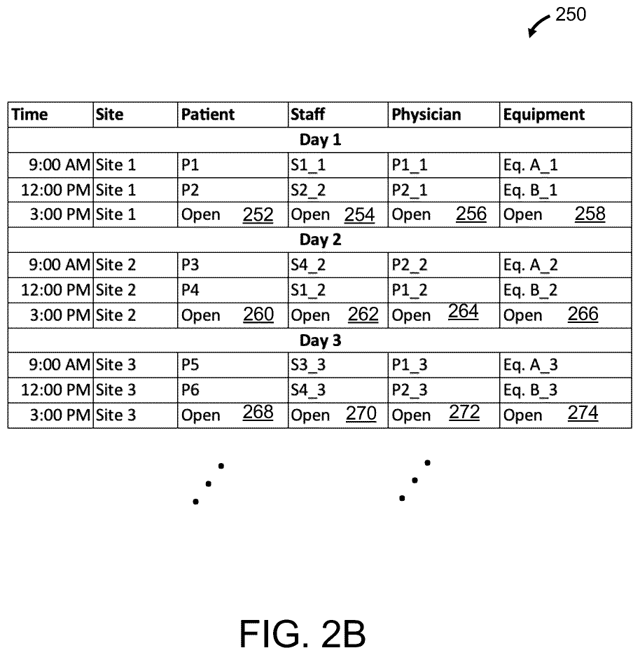

FIG. 2A is an example schedule 200 generated by the system of FIGS. 1A-1B. The schedule 200 may be generated by scheduling system 102 to schedule 20 patients across 6 clinics at 6 locations (e.g., sites), for example. Although 6 clinics across 6 locations are used as an example, any number of clinics or locations may be used. In this example, each location includes 3 pieces of dialysis equipment, 4 staff members, and 2 physicians. Each appointment for each patient occurs at a single site for 3 hours and includes the patient, one staff member, and one optional physician and at least one piece of equipment. In this example, patient times are not overlapped, but given the number of staff, physicians and equipment, overlap could instead occur.

Here, row one 202 of the schedule includes a first patient (P1) is scheduled at site 1 at 9:00 AM with staff 1, physician 1, and equipment A at site 1. Similarly, patients P2 and P3 are scheduled at site one in the next two rows. Next, sites 2, 3, 4, 5, and 6 are scheduled for patients P4 through P20.

FIG. 2B is another example schedule 250 generated by the system of FIGS. 1A-1B. The schedule 250 is a partial schedule for the purpose of brevity. In the schedule 250, the scheduling system 102 generated open time slots 252-274 in the schedule to ensure that time slots remain open for last minute bookings and/or rescheduling. The open time slots may account for staff, physicians, and equipment that may be scheduled for patients at a future time.

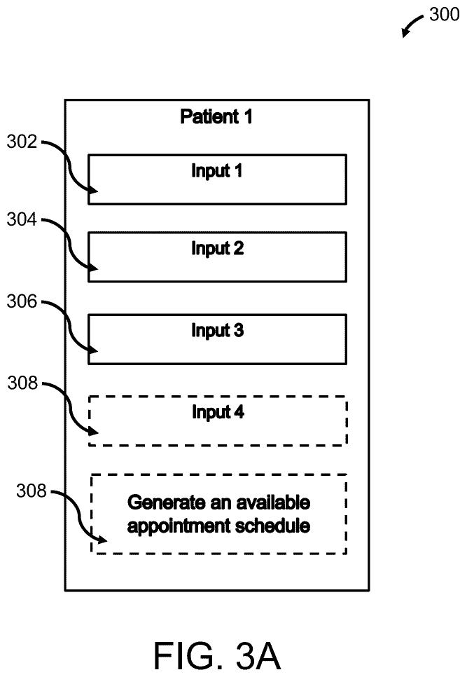

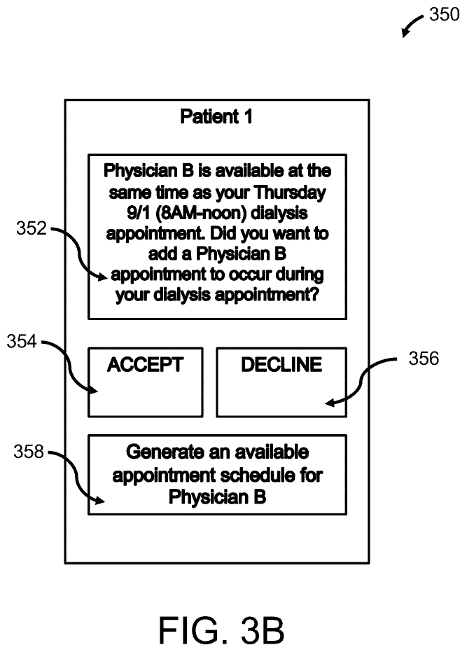

FIG. 3A is an example user interface 300 generated by the system of FIGS. 1A-1B. The UI 300 may be presented to a patient on a patient device (e.g., wearable device 132 or a computing device associated with the patient). In some embodiments, the UI 300 may be part of an app (e.g., apps 177) provided by scheduling system 102 to a patient, staff member, or physician. The app 177 may be used to enter inputs. In some embodiments, the inputs may be obtained from data available to scheduling system 102 and may be automatically obtained, rather than entered by any user.

In this example, the app 177 may be provided to patient 1 where any or all of an input 302, an input 304, an input 306, and an optional input 308 may be received from patient 1 or otherwise obtained by system 102 as described elsewhere herein. In some embodiments, a single input may be provided. In some embodiments, two inputs may be provided. In some embodiments, three inputs may be provided. In some embodiments, four or more inputs may be provided.

Upon one or more inputs being provided with respect to UI 300, an optional selectable button 310 may be provided to allow the patient to generate an available appointment schedule. In some embodiments, the button 310 is not provided and instead system 102 automatically generates an available appointment schedule based on available input provided (e.g., input 302, input, 304, input 306, and/or input 308).

FIG. 3B is an example user interface 350 generated by the system of FIGS. 1A-1B. The UI 350 may be presented to a patient on a patient device (e.g., wearable device 132 or a computing device associated with the patient) in response to determining that the patient has a dialysis appointment at a first time and is also in need of a physician visit. In some embodiments, the UI 350 may be part of an app (e.g., apps 177) provided by scheduling system 102 to a patient, staff member, or physician. The app 177 may be used to enter inputs for accepting suggested appointments and/or generating additional possible appointment schedules.

As shown, a message is provided indicating possible add on appointments for the patient 1. For example, the message generator 160 may have generated the message containing an indication having one or more appointment indicators 352. In this example the indicator 352 includes a message that “Physician B is available at the same time as your 9/1 (8 AM-noon) dialysis appointment. Did you want to add a Physician B appointment to occur during your dialysis appointment?” The indicator 352 may instead include any number of appointment times, questions, or the like. For example, the message generator 160 may generate and transmit one or more dates and time periods to a scheduling system (e.g., scheduling system 102 or another external system) of one or more physicians for treating a secondary condition of the patient and may determine an overlap (e.g., via scheduling engine 112) between the schedule of the one or more physicians (e.g., via calendars 152) and the one or more dates and time periods. In response to finding the overlap, the message generator 160 may send the proposed dates/time periods via a UI or message including UI content (e.g., such as UI 350). The patient may receive the UI 350, and accept 354 or decline 356 the proposed appointment. In some embodiments, the appointment is a virtual appointment between the patient and the one or more physicians during the dialysis machine usage by the patient. In some embodiments, the appointment is an in-person appointment between the patient and the one or more physicians during the dialysis machine usage by the patient.

If the patient accepts 354 the proposed appointment (or selects one of the appointments from the proposed appointments), then the scheduling engine 112 may schedule an appointment with the one or more physicians during the one or more dates and time periods. If the patient declines 356 the proposed appointments or selects another option, other actions may be performed by system 102. For example, UI 350 includes a selection 358 to generate an available appointment schedule for Physician B. If the patient selects selection 358, then the scheduling system 102 may use physician schedules/profiles 110 and/or calendars 152 to generate the appointment schedule for Physician B and provide such a schedule to the patient.

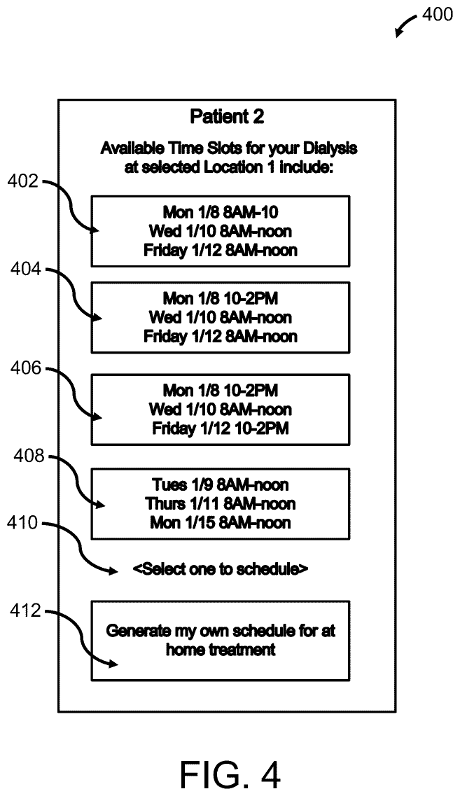

FIG. 4 is another example user interface 400 generated by the system of FIGS. 1A-1B. The UI 400 may be presented to a patient on a patient device (e.g., wearable device 132 or a computing device associated with the patient) in response to determining that the patient has a prescription for dialysis treatment. In some embodiments, the UI 400 may be part of an app (e.g., apps 177) provided by scheduling system 102 to a patient, staff member, or physician. The app 177 may be used to provide access to scheduling system 102 for a patient, staff member, or physician and to enter inputs for accepting suggested appointments and/or generating additional possible appointment schedules.

As shown, a message is provided indicating available time slots for dialysis for patient 2 at a selected location 1. The time slots include indicators 402, 404, 406, and 408 depicting appointment series of 2 or 4 hour blocks. The patient 2 may select from one of the appointment series to trigger system 102 to schedule the appointments, as indicated by text indicator 410. In addition, an indicator 412 to generate the patient's own schedule for at home treatment is also provided to trigger the system 102 to offer appointments in which equipment and staff and/or physicians are available for home-based visits and treatment.

### EXAMPLE PROCESSES

FIG. 5 illustrates a flow diagram of an example process 500 for rescheduling patients. In some embodiments, the process 500 may be performed by scheduling system 102 and in particular, by optimizer engine 116 to assess particular patient risks and/or communication engine 114 to detect particular system or patient events. Ranking engine 148, ML models 150, calendars 152, and/or virtual UI generator 154 may also be used in one or more steps of the process 500. In some embodiments, the process 500 may be carried out by one or more ML algorithms using ML models 150, for example.

At block 502, the process 500 includes detecting an event. For example, the delay risk module 170 may determine that a first patient cancelled or missed an appointment and because of the cancellation (or other input), the first patient may be at a higher hospitalization risk than a second patient on the existing schedule. In one non-limiting example, the event detector 162 and/or the patient data detector 164 may detect a medical anomaly in data captured or received from the first patient and/or an event indicating distress or medical decline of the first patient. In response, the system 102 may attempt to reschedule the second (and lower hospitalization risk) patient in order to provide a time slot for the first patient to mitigate the hospitalization risk. In some embodiments, the detected event may be received in an app 177 accessed by the patient. The process 500 may detect such events and proceed to assist the patient in rescheduling one or more appointments.

For example, at block 504, the process 500 may include accessing one or more scheduled patient lists that fit a predefined time period allotted for the first patient in a prescribed dialysis protocol associated with the first patient. The scheduling engine 112 may use analysis module 140 and/or ML models 150 to assess whether an opening can be made in the schedule for the first patient to receive medical care (e.g., dialysis, physician care, etc.). For example, the optimizer engine 116, and in particular, the patient risk module 172 may determine a hospital risk for patients in the patient lists, at block 506, to assess whether any patient can delay a scheduled appointment and remain a low risk of hospitalization. The patient risk module 172 can determine any number of appointments that may be rescheduled for another patient in order to allow the first patient to avoid a hospitalization risk.

At block 508, the process 500 includes selecting one or more appointments associated with a patient with a low hospitalization risk. For example, the patient risk module 172 may select one or more appointments associated with one of the low risk patients determined herein. The selected one or more appointments may be candidate choices for rescheduling. In some embodiments, selecting the one or more appointments for reschedule may include generating and sending messages to respective patients that may be rescheduled to request whether a respective patient would tolerate or accept such a rescheduling or to entirely reschedule the respective patient, as indicated by arrow 509.

At block 510, the process 500 includes offering a selected appointment to the first patient. For example, the scheduling system (e.g., the message generator 160) may generate a message for the patient associated with the detected event (i.e., the first patient) to offer the selected appointment(s).

At block 512, the process 500 includes detecting whether one or more of the offered appointments has been accepted by the first patient. For example, the event detector 162 can detect if the first patient accepted an appointment. If the first patient did not accept the appointment, then the system 102 can return to block 508 to generate and select additional appointment candidates to offer to the first patient. If the first patient did accept one or more of the offered appointments, the system 102 may schedule the first patient, at block 514. For example, the scheduling engine 112 may generate the appointment in the system 102 for any staff, equipment, and/or physicians and may schedule the first patient at the time(s) and day(s) of the offered one or more appointments.

At block 516, the process 500 includes offering one or more rescheduling appointments for the patient(s) in which an appointment was selected for rescheduling. For example, the message generator 160 may generate and transmit a message to the second patient (having a lower hospitalization risk than the first patient). The message may include and/or otherwise provide for alternative appointment date(s)/time(s) for the second patient to accommodate a new appointment for the second patient while still following the prescribed treatment time period associated with the second patient.

In operation of process 500, the system 102 may have scheduled a number of appointments for any number of patients. At some point in time, the system 102 may receive a cancellation input (e.g., from the first patient) indicating cancellation of a previously schedule first time period on a first date. The system 102 may generate and output to the first patient, a health impact message when a second time period on a second date within the predefined time window is not selected. In this example, the first patient may have cancelled due to a personal scheduling conflict. The system 102 can determine that this would be a health risk to the first patient and may automatically attempt to reschedule the cancelled appointment for another time that is still within the prescribed time window for dialysis treatments in a week, for example. In particular, the message generator 160 may function with the recommendation generator 142 to generate and recommend an output of an appointment at a second time period on a second date within the predefined time window of the first patient. Such an assessment may ensure that the patient is offered a new appointment that is still within treatment guidelines for the week, for example. The rescheduling process may involve additional patients in the event that the schedule is full. For example, additional messages and rescheduling tasks can be sent to other patients with a lower hospitalization risk than the first patient. The messages may provide alternative appointment dates for the second patient to accommodate the second time period on the second date for the patient. (e.g., as described in detail herein).

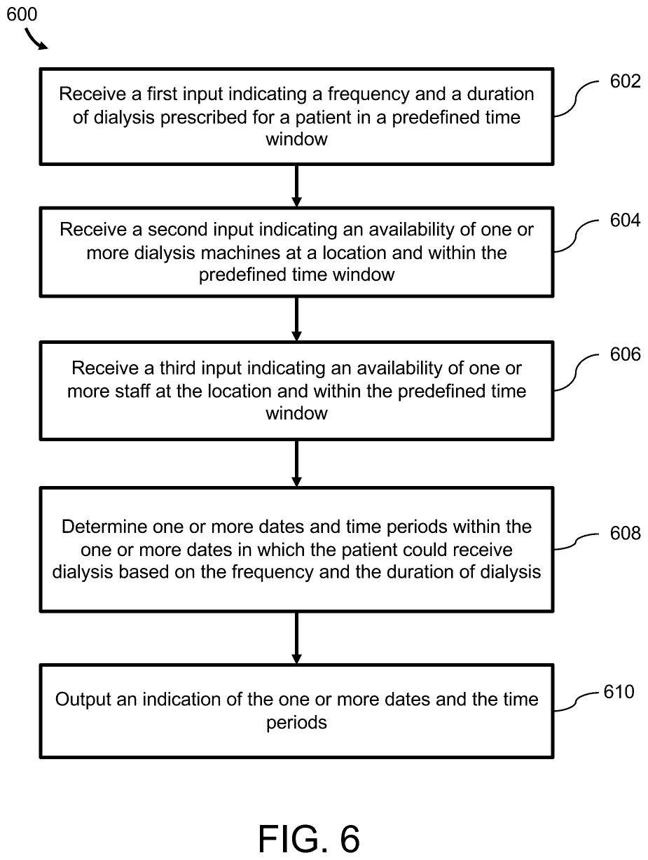

FIG. 6 is a flowchart of an example process 600 for generating scheduling options that optimize dialysis machine usage. In some embodiments, the process 600 may be performed by scheduling system 102 and in particular, by scheduling engine 112, optimizer engine 116, and communication engine 114. Ranking engine 148, ML models 150, calendars 152, and/or virtual UI generator 154 may also be used in one or more steps of the process 600. In some embodiments, the process 600 may be carried out by one or more ML algorithms using ML models 150, for example.

At step 602, the process 600 includes receiving a first input (e.g., input 302) indicating a frequency and a duration of dialysis prescribed for a patient in a predefined time window. For example, a physician, a staff member, or a patient may provide the first input in a user interface (e.g., UI 300) provided by the scheduling system 102. In some embodiments, the first input may be obtained by system 102 automatically by accessing calendars 152 and/or patient data 104 and/or schedules 122.

In some embodiments, the first input may include a prescription in text or image format. In some embodiments, the first input may include data obtained from dropdown elements, radial selections, and/or text fields presented in UI 300 and accessible to system 102 upon completion of patient entry of inputs, for example. In some embodiments, the first input may be a provider name or location in which treatments are typically received by the patient. The entered provider name may trigger access by system 102 to a provider database that may provide patient information (e.g., prescription) according to user preferences and/or user permissions. The access to the provider database may enable system 102 to query the database to obtain patient details and/or prescription information and thus provide the system 102 with reference to a number of treatments that should occur within a predefined period indicated by the prescription. In this way, the system 102 ensures that a patient does not extend or shorten the time of the treatments or the time between treatments. In some embodiments, the system 102 may use a provider database to verify a prescription based on the patient data and may do so without the input of the provider's name or location.

Although a single patient may be referenced in the processes described herein, the term “patient” may refer to a plurality of patients in which each patient has a corresponding individualized frequency and duration of dialysis in an individualized, predefined time window. For example, when the processes 500 and/or 600 are indicated to access, modify, or query data about a single patient, data for any number of patients may instead be accessed, modified, or queried. Such access to multiple patients can ensure that process 500 and process 600 may schedule, reschedule, and query any number of patients in real time with scheduling system 102. In such examples, the system 102 may offer a way for staff/schedulers/providers to query the appointments and calendars 152 in the system while also offering patients a way to access data to find convenient appointments according to prescription timing. Thus, the scheduling system 102 provides an automated and simultaneous way to query, schedule, and reschedule appointments on the staff/schedulers/providers side and the patient side in real time.

At step 604, the process 600 includes receiving a second input (e.g., input 304) indicating an availability of one or more dialysis machines at a location and within the predefined time window. For example, a physician or a staff member may provide the second input in a user interface (e.g., UI 300) provided by the scheduling system 102. In some embodiments, the second input may be obtained by system 102 automatically by accessing calendars 152 and/or equipment schedules 108 and determining availability windows of time for the equipment at a particular location. Such availability windows may be compared and matched to the predefined time window associated with the patient prescription to find an overlap in availability between both equipment and patent schedules. Accordingly, the input may be obtained by system 102 and presented in UI 300 as options in which to select particular dialysis machines or particular locations. The second input and subsequent presentation of options of equipment can be based on scheduled appointments, scheduled equipment maintenance, staff availability, etc.

At step 606, the process 600 includes receiving a third input (e.g., input 306) indicating an availability of one or more staff at the location and within the predefined time window. For example, a physician or a staff member may provide the third input in a user interface (e.g., UI 300) provided by the scheduling system 102. In some embodiments, the third input may be obtained by system 102 automatically by accessing calendars 152 and/or physician schedules/profiles 110 and/or staff schedules/profiles 106. The third input may include physician schedules, staff schedules, physician or staff preferences, vacation schedules, or the like.

In some embodiments, the third input may be obtained by system 102 automatically by accessing calendars 152 and/or physician schedules/profiles 110 and/or staff schedules/profiles 106 and determining availability windows of time for the staff and/or physician that overlaps availability for the equipment and the patient. For example, the physician and/or staff availability windows may be compared and matched to the predefined time window associated with the patient prescription to find an overlap in availability between staff/physician and patent schedules. Accordingly, the third input may be obtained by system 102 and presented in UI 300 as options in which to select particular staff and/or physicians.

At step 608, the process 600 includes determining one or more dates and time periods within the one or more dates in which the patient could receive dialysis based on the frequency and the duration of dialysis. For example, the analysis module 140 may assess the first input, the second input, and the third input to determine suggestions of appointments or appointment series that may be presented to one or more patients. The suggestions may include one or more dates and the time periods that are aligned with the availability of the one or more dialysis machines and the availability of the one or more staff. In some embodiments, the process 600 may determine the availability of the one or more staff based on a schedule (e.g., staff schedules/profile 106) for each staff and one or more predefined staffing ratios. For example, predefined staffing ratios may indicate a staff-to-patient ratio of about 1 to 2; about 1 to 3, about 1 to 4; or about 1 to 5 to ensure patient safety and positive patient outcomes. Thus, scheduling suggestions can be based at least in part on staffing ratios.

In some embodiments, the second input associated with location may include a plurality of locations, such that the one or more dialysis machines include any number of dialysis machines. In some embodiments, the third input associated with one or more staff may include any number of technicians available to operate any of the dialysis machines. In this way, a user can select particular staff and particular physicians based on the availability and details for each location and/or piece of equipment. In such examples, the process 600 may further include determining the one or more dates and time periods in which the patient could receive dialysis at a particular one of the plurality of locations and the time periods within the one or more dates in which the patient is recommended to receive dialysis based on the frequency and the duration of dialysis. Thus, the system 102 may narrow location options to a single location based on any one or more of a patient address, traffic information, patient delay, patient preferences, and/or physician requests. In some embodiments, the system 102 may narrow location options to two or three locations based on any one or more of a patient address, traffic information, patient delay, patient preferences, and/or physician requests.

At step 610, the process 600 includes outputting an indication of the one or more dates and the time periods in which the patient may wish to schedule appointments. The indication may include a message. For example, the indication may include an email, a calendar invite, an indicator in a UI (e.g., UI 400), a text message, an app based UI or indication, a phone call with prompts, etc., or the like. In some embodiments, the indication may include an optimized schedule for the patient for a week, a month, a year, etc. The optimized schedule may be based on health risks, cost risks, and/or other input that may affect patient adherence to a dialysis schedule. The patient may accept and/or decline suggested indications of appointments, as described in detail elsewhere herein. For example, if the patient wishes to accept one of the offered appointments, the patient may select the appointment. In response, the scheduling system 102 may receive the selection as input, for example, to select a first time period of the one or more time periods on a first date of the one or more dates.

In some embodiments, the process 600 further includes receiving a fourth input (e.g., optional fourth input 308) indicating one or more of: a drive time for the patient, a weather input of the location, and a delay risk metric. The fourth input may be automatically obtained by system 102 or entered by a patient into a UI generated by system 102. In some embodiments, the fourth input may include a drive time for the patient based on traffic input 130 and/or location tracking information and/or address information associated with patient data 104. In some embodiments, the fourth input may include a weather input associated with the location of suggested appointments and such input may be obtained by weather input 128. In some embodiments, the fourth input may include a delay risk metric indicating that a patient may be at risk of arriving late to an appointment. The delay risk metric may be calculated based on one or more of the traffic data 130, a historical appointment attendance for the patient, a schedule 108 of one or more additional dialysis machines at the location, or a schedule 106 of one or more additional staff at the location. The delay risk metric may be used to reschedule the patient and/or trigger modifications at the site of the appointment for staff, physicians, and/or equipment. For example, the fourth input may be used to enable the scheduling system 102 (i.e., the scheduling engine 112) to automatically update the one or more dates and the time periods for the patient.

In some embodiments, the fourth input includes input indicating one or both of: a fluid load or a lifestyle metric of the patient. For example, the wearable device 132 may be polled to obtain such information. In some embodiments, the patient may provide specific fluid load or lifestyle metric input (e.g., eating habits between dialysis appointments, alcohol intake, controlled substance intake, exercise schedule, etc.) in an app associated with (or in communication with) scheduling system 102. In response to receiving such input, the scheduling system 102 may automatically update the one or more dates and the time periods according to the received fluid load or lifestyle metric(s).

In some embodiments, the received fluid load may be based on one or both of: a historical weight gain of the patient or a historical fluid removal rate during dialysis of the patient. Such information may be obtained from an internet-connected scale that captures the weight of the patient over time, received from user input, or accessed from an EMR of the patient. The weight over time may be used by system 102 to determine the historical weight gain of the patient. Such weight assessments and fluid levels may be used as a basis in which to generate additional appointments, reschedule appointments, cause health related messages to be sent to the patient, etc. For example, the scheduling system 102 may receive a fourth input indicating a volume of fluid removed from the patient during a previous time period on a previous date. The system 102 may determine whether scheduling is to be changed according to the newly received input and in response to such a determination, may automatically update the one or more dates and the time periods.

In some embodiments, receiving the fourth input includes receiving historical data that includes appointment frequency per time period for a physician. Such input may be used to automatically update the one or more dates and the time periods to fall within a window of higher appointment frequency for the physician. This can ensure that a physician can time block appointments at particular locations to avoid increasing costs pertaining to physician travel time, physician wait time, and physician down time.

In some embodiments, the received inputs described herein may be received in one or more apps 177 communicatively coupled to the scheduling system 102. Patients, staff, and physicians may have access to such apps 177.

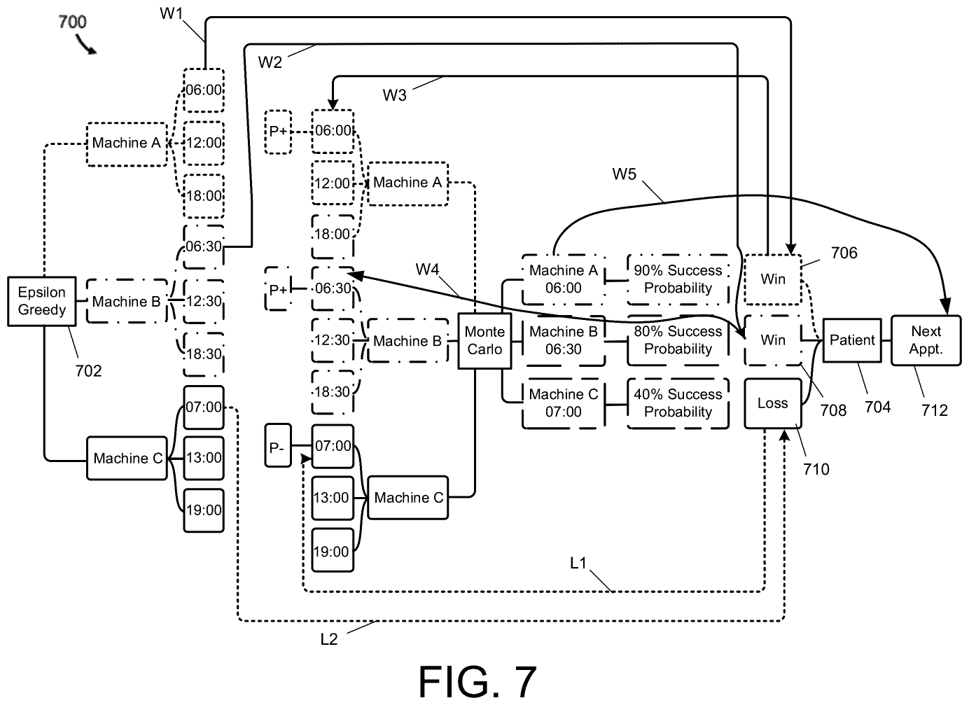

FIG. 7 is an example appointment selection process 700 for use with the system of FIGS. 1A-1B. An Epsilon-Greedy node 702 may utilize one or more ML models to determine whether predicted scheduling recommendations will be accepted by a patient (e.g., a win) or whether such predicted scheduling recommendations will be rejected by the patient (e.g., a loss).

In this example, machine A, machine B and machine C may each represent a dialysis machine (e.g., or other equipment). Each machine is depicted with three corresponding time blocks available for machine utilization. While three time blocks are shown, any number of time blocks may instead be substituted (e.g., 4, 5, 6, 7, 8, 9, 10, 11, 12 . . . 24 time blocks).

The arrows W1, W2, W3, W4, L1, and L2 that extend from a time block to the win (win 706, win 708) or loss 710 for the patient represent attempts by the algorithm to determine one or more ideal appointment windows for the patient. The W1, W2, W3, and W4 arrows represent appointments that were considered Wins; the L1 and L2 arrows represent appointments that were considered losses.

In operation, the system 102 may suggest recommended next appointments to a patient or to a user that sets a patient's schedule. The suggested next appointments are derived using one or more Monte Carlo simulations, as described elsewhere herein, which may employ algorithms utilized by the Epsilon-Greedy node 702. The system 102 selects future appointments that have a high probability of being wins and provides the selections as suggestions to the patient or other user setting a schedule. In this example, the suggested appointment is shown by W5, which is presented to the user/patient as an option for the next appointment 712 based on being determined by system 102 to have a 90% probability of success of being selected by the user/patient. Any choice selected by the patient or the user setting the schedule may be considered in future iterations of the algorithm. For example, selections may be considered weighting factors for any given machine or time window configuration in future appointment suggestions.

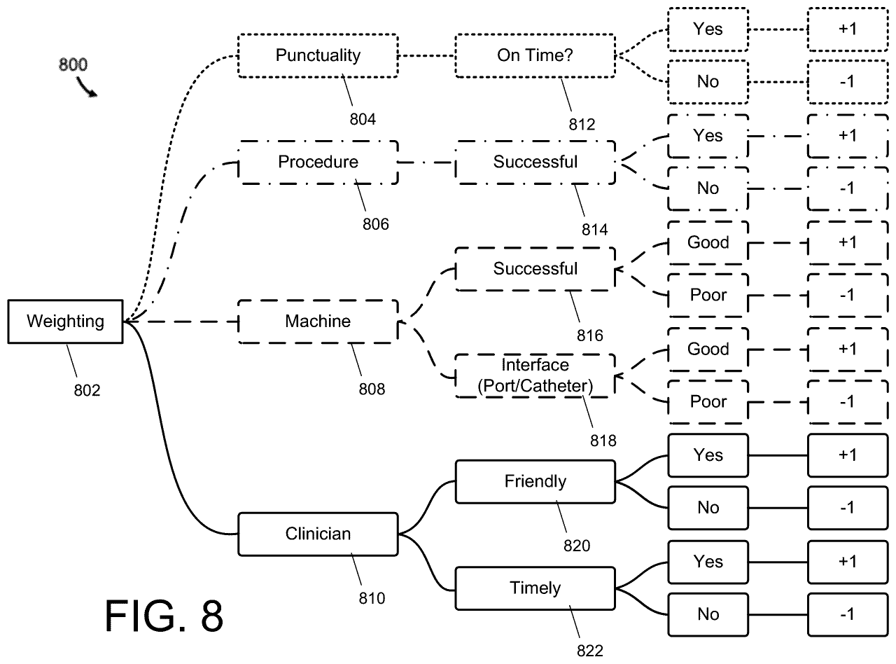

FIG. 8 is an example weighting process 800 for use with the system of FIGS. 1A-1B. The process 800 may assign numerical values to various factors, such as punctuality metrics, procedure success metrics, machine performance metrics, nurse interaction metrics, and the like. These values may be combined to generate an overall patient experience score.

In general, the process 800 may provide a quantitative approach to evaluating patient experience. In some embodiments, the process 800 may provide customization of weightings based on specific priorities or preferences. In some embodiments, the process 800 may be used to identify areas for improvement in patient care.

In a non-limiting example, the process 800 may assign weights based on punctuality 804, procedure 806, machine 808, and/or clinician 810. The weights generated by performing weighting 802 may provide an overall patient experience score. The punctuality 804 may be weighted based on whether a patient arrived on time 812 for one or more prior appointments. A positive weight may be assigned for on-time arrivals, and a negative weight may be assigned for late arrivals.

In a non-limiting example, the process 800 may assign weights based on a particular procedure being successful 814 or not. For example, the system 102 may evaluate a success of a dialysis procedure. A positive weight may be assigned for successful procedures, and a negative weight may be assigned for unsuccessful procedures.

In a non-limiting example, the process 800 may assign weights based on a machine successfully operating and/or factors such as flow rate and interface quality 818 of a port/catheter, for example. Positive weights may be assigned for good performance, and negative weights may be assigned for poor performance.

In a non-limiting example, the process 800 may assign weights based on performance or demeanor of a clinician (e.g., nurse, doctor, technician, etc.). For example, the system 102 may assesses the friendliness 820 and/or timeliness 822 of a clinician. Positive weights may be assigned for positive interactions, and negative weights may be assigned for negative interactions.

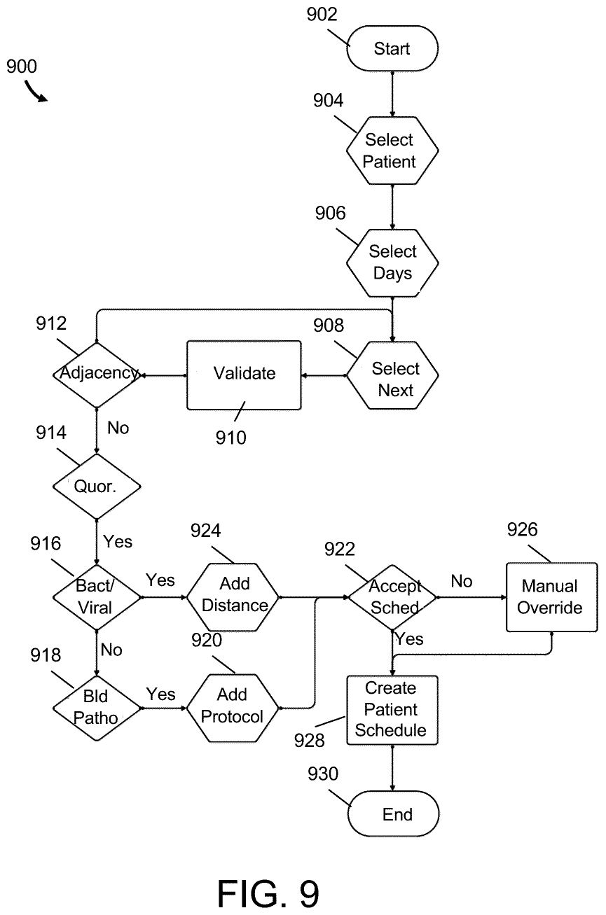

FIG. 9 is a flow chart of an example scheduling process for use with the system of FIGS. 1A-1B. The process 900 may additionally function to carry out some or all aspects of block 608 of FIG. 6. In some embodiments, the process 900 functions to schedule patient appointments. For example, the process 900 may be used for dialysis scheduling but can additionally or alternatively be used for any suitable applications, clinical or otherwise. The process 900 can be configured and/or adapted to function for any suitable application for scheduling patients. In general, the process 900 may prioritize patient safety by considering quarantine restrictions and implementing appropriate protocols while offering flexibility by allowing for manual schedule adjustments.

The process 900 may be performed by system 102. The system 102 may begin by selecting a patient and then may proceed to select available days of the week for scheduling. The system 102 may validate the schedule, considering factors such as adjacency issues and quarantine restrictions. If issues arise, the system 102 may add distancing protocols or manually override the schedule. If no issues are identified, the system 102 may generate a patient schedule and ends the scheduling process.

At block 902, the process 900 may include initiating the scheduling of a patient with equipment and/or clinician(s). At block 904, the system 102 may select a patient for scheduling. At block 906, the system 102 may identify available days of the week for the selected patient. At block 908, the system 102 may select a next available time block. At block 910, the system 102 may validate the schedule by checking for any scheduling conflicts. For example, the system 102 may check for adjacency issues, at block 912, quarantine issues, at block 914, bacterial or virulence issues, at block 916, and/or blood borne pathogen issues, at block 918. If there are adjacency issues, the process 900 returns to select a next available time block at block 908. If there are no adjacency issues, then the process 900 determines whether there are quarantine issues and/or bacterial virulence issues. If either is true for the selected patient, then process 900 may indicate to add distancing to the appointment, at block 924. If there are neither are true, then the process 900 may determine whether blood borne pathogen issues are present for the selected patient, at block 918. If there are no blood pathogen issues, then process 900 may skip to block 922. If there are blood pathogen issues, the process 900 may move to block 920 to add Medicare or other pathogen protocols to the appointment.

The determined appointment may be provided to the patient for approval or denial, as described elsewhere herein. Any number of determined/recommended appointments may be sent to the patient to provide options in which to allow the patient to choose from many recommended appointment times/days (or sets of appointments). If the patient rejects a suggested appointment (or set of appointments), then the process 900 may enable the user to manually override schedule adjustments, at block 926. If the patient accepts a suggested appointment (or set of appointments), then the process 900 generates the patient schedule according to the selected days/times, at block 928, which ends scheduling, at block 930.

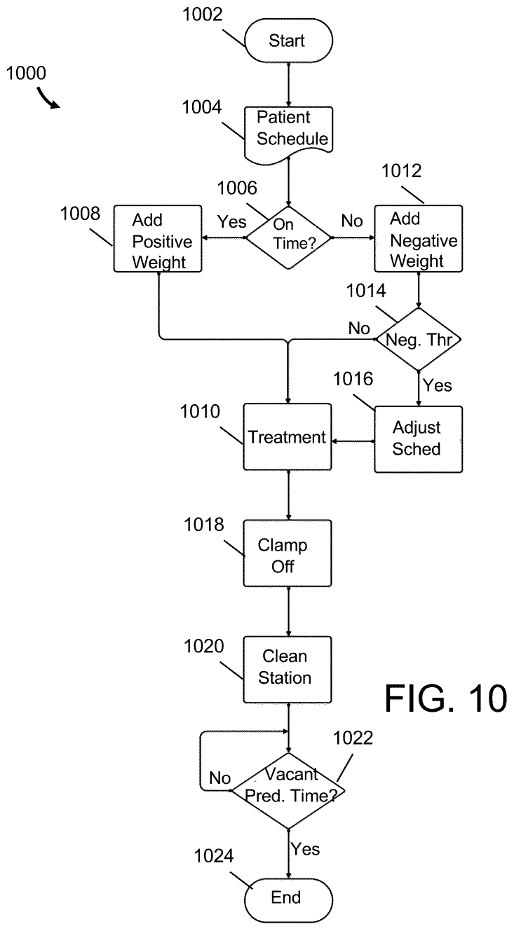

FIG. 10 is a flow chart of an example weighted scheduling process 1000 for use with the system of FIGS. 1A-1B. The process 1000 may prioritize patient punctuality by adjusting schedules based on negative weights and/or positive weights. In some embodiments, the process 1000 ensures efficient station utilization by monitoring vacancy time. In some embodiments, the process 1000 functions to schedule patient appointments. For example, the process 1000 may be used for dialysis scheduling but can additionally or alternatively be used for any suitable applications, clinical or otherwise. The process 1000 can be configured and/or adapted to function for any suitable application for scheduling patients.

The process 1000 illustrates a system and method for managing patient treatment and scheduling. The process 1000 may begin by initiating the scheduling process and assessing an on-time status (e.g., using punctuality metrics associated with prior user visits, traffic data, weather data, etc.). The process 1000 may use the punctuality metrics to adjust the schedule accordingly based on predefined negative thresholds, for example.

At block 1002, the process 1000 may include assessing one or more prior visits according to a start time, at block 1002, and a patient schedule, at block 1004. The assessment may include determining whether the patient was on time for one or more prior appointments, at block 1006. If the patient was on time for a particular appointment, the process 1000 may add a positive weight associated with the timing of the prior appointment, at block 1008. The process 1000 may continue to schedule the treatment for the patient at the proposed time, at block 1010.

If the patient was not on time for the particular appointment, then the process 1000 may add a negative weight associated with the timing of the prior appointment, at block 1012. Each prior appointment can be assessed in a similar fashion. The process 1000 may determine whether any added negative weight pushes an overall weighting of any particular appointment over a negative threshold, at block 1014.

Examples of a negative threshold may include a consistently missed appointment window eventually losing enough weight that it falls out of the recommendation list. The overall sensitivity of the system 102, which triggers this behavior may be a modifiable parameter, consistent with an implementation of Epsilon-Greedy algorithms. In Epsilon-Greedy terms, the system 102 may shift from an exploitation state to a discovery state, seeking a more efficient appointment window aggressively instead of continuing to schedule an unfruitful appointment time.

If the added negative weight pushes the overall weighting over the negative threshold, then the process 1000 may adjust the schedule to suggest a different appointment time (or set of appointment times) to accommodate for the likelihood of the user being tardy to future appointments. Upon adjusting the schedule, a user (e.g., the patient) may select to approve the adjusted schedule and the process 1000 may schedule the treatment at the accepted time, at block 1010. Once the treatment is scheduled, time may be further scheduled to either or both clamp-off the patient, at block 1018, and clean any workstation, at block 1020.

At block 1022, the process 1000 may determine whether the treatment station is or will be vacant for a time at or above a predetermined time span (e.g., 15 minutes, 30minutes, 45 minutes, 1 hour, 1.5 hours, 2 hours, 4 hours, etc.). If the station will not be vacant for a time at or above the predetermined time span, the process 1000 may again check vacancy until the treatment station is to be vacant for a time at or above the predetermine time span. If the station will be vacant for the predetermined time span or more than the predetermined time span, the process 1000 may end the session, at block 1024.

The systems described herein may generate requisite warnings to protect the information contained in data accessed and/or stored by such systems in compliance with the Health Insurance Portability and Accountability Act (HIPPA). Accordingly, the systems and methods described herein provide compliance with HIPPA, and other privacy requirements regarding patient medical data, or other personal information. For example, the processes 500, 600, 700, 800, 900, and 1000 are executed in such a fashion to be HIPPA compliant and requisite warnings are provided to protect the information contained in system 102 in compliance with HIPPA.

The systems and methods of the implementations described herein and/or variations thereof can be embodied and/or implemented at least in part as a machine configured to receive a computer-readable medium (or computer program product) storing computer-readable instruction. The instructions are executed by computer-executable components integrated with the system and one or more portions of the hardware processor 178 communicatively coupled computing device. The computer-readable medium (or computer program product) can be stored on any suitable computer-readable media (e.g., memory 180) such as RAMs, ROMs, flash memory, EEPROMs, optical devices (e.g., CD or DVD), hard drives, floppy drives, or any suitable device. The computer-executable component can be a general or application-specific hardware processor, but any suitable dedicated hardware or hardware/firmware combination can alternatively or additionally execute the instructions.

A computer program product can be tangibly embodied in an information carrier. The computer program product may also contain instructions that, when executed, perform one or more methods and/or computer-implemented methods described herein. The information carrier may be a computer-or machine-readable medium, such as the memory 180, or other storage associated with system 102 and/or wearable device 132 and/or processors 178. In general, the scheduling engine 112, communication engine 114, and optimizer engine 116 and their respective modules, generators, or detectors may each be executed to carry out the steps and algorithms described herein using one or more processors 178 and one or more memory 180.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used in the description and claims, the singular form “a”, “an” and “the” include both singular and plural references unless the context clearly dictates otherwise. For example, the term “signal” may include, and is contemplated to include, a plurality of signals. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5 percent, 1 percent or 0.1 percent. All numerical ranges provided herein are inclusive of the stated start and end numbers. The term “substantially” indicates mostly (i.e., greater than 50%) or essentially all of a device, substance, or composition.

The term “horizontal” as used herein is defined as a plane parallel to the conventional plane or surface of an element, regardless of its orientation. The term “vertical” refers to a direction perpendicular to the horizontal as just defined. Terms, such as “on”, “above”, “below”, “bottom”, “top”, “side” (as in “sidewall”), “higher”, “lower”, “over”, and “under”, are defined with respect to the horizontal plane.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Implementations defined by each of these transitional terms are within the scope of this disclosure.

The examples and illustrations included herein show, by way of illustration and not of limitation, specific implementations in which the subject matter may be practiced. Other implementations may be utilized and derived therefrom, such that structural and logical substitutions and changes may be made without departing from the scope of this disclosure. Such implementations of the inventive subject matter may be referred to herein individually or collectively by the term “invention” merely for convenience and without intending to voluntarily limit the scope of this application to any single invention or inventive concept, if more than one is in fact disclosed. Thus, although specific implementations have been illustrated and described herein, any arrangement calculated to achieve the same purpose may be substituted for the specific implementations shown. This disclosure is intended to cover any and all adaptations or variations of various implementations. Combinations of the above implementations, and other implementations not specifically described herein, will be apparent to those of skill in the art upon reviewing the above description.

What is claimed is:

1. A computer-implemented method for optimizing dialysis machine usage, the method comprising:
receiving a first input indicating a frequency and a duration of dialysis prescribed for a patient in a predefined time window;
receiving a second input indicating an availability of one or more dialysis machines at a location and within the predefined time window;
receiving a third input indicating an availability of one or more staff at the location and within the predefined time window;
determining one or more dates and time periods within the one or more dates in which the patient could receive dialysis based on the frequency and the duration of dialysis, wherein the determined one or more dates and the time periods are aligned with the availability of the one or more dialysis machines and the availability of the one or more staff; and
outputting an indication of the one or more dates and the time periods.

2. The computer-implemented method of claim 1, further comprising:
receiving a fourth input indicating one or more of: a drive time for the patient, a weather input of the location, and a delay risk metric; and
automatically updating the one or more dates and the time periods based on the fourth input.

3. The computer-implemented method of claim 2, wherein the delay risk metric is calculated based on one or more of: traffic data, a historical appointment attendance for the patient, a schedule of one or more additional dialysis machines at the location, or a schedule of one or more additional staff at the location.

4. The computer-implemented method of claim 1, further comprising receiving a selection input indicating a first time period of the time periods on a first date of the one or more dates.

5. The computer-implemented method of claim 4, further comprising:
receiving a cancellation input indicating cancellation of the first time period on the first date; and
outputting a health impact message when a second time period on a second date within the predefined time window is not selected.

6. The computer-implemented method of claim 4, further comprising:
receiving a cancellation input indicating cancellation of the first time period on the first date; and
outputting a second time period on a second date within the predefined time window.

7. The computer-implemented method of claim 6, further comprising transmitting a message to a second patient having a lower hospitalization risk than the patient, wherein the message provides alternative appointment dates for the second patient to accommodate the second time period on the second date for the patient.

8. The computer-implemented method of claim 1, further comprising receiving a fourth input indicating one or both of: a fluid load or a lifestyle metric of the patient; and automatically updating the one or more dates and the time periods based on the fourth input.

9. The computer-implemented method of claim 8, wherein the fourth input is received from a wearable device.

10. The computer-implemented method of claim 8, wherein the fluid load is based on one or both of: a historical weight gain of the patient or a historical fluid removal rate during dialysis of the patient.

11. The computer-implemented method of claim 10, further comprising:
receiving, from an internet-connected scale, a weight of the patient over time; and
determining the historical weight gain of the patient based on the received weight.

12. The computer-implemented method of claim 1, further comprising:
receiving a fourth input indicating a volume of fluid removed from the patient during a previous time period on a previous date; and
automatically updating the one or more dates and the time periods based on the fourth input.

13. The computer-implemented method of claim 1, wherein the patient comprises a plurality of patients, each patient having an individualized frequency and duration of dialysis in an individualized, predefined time window.

14. The computer-implemented method of claim 1, wherein the location comprises a plurality of locations, such that the one or more dialysis machines comprise a plurality of dialysis machines and the one or more staff comprise a plurality of technicians.

15. The computer-implemented method of claim 14, wherein the determining further comprises determining the one or more dates at one of the plurality of locations and the time periods within the one or more dates in which the patient is recommended to receive dialysis based on the frequency and the duration of dialysis.

16. The computer-implemented method of claim 1, wherein the determining is performed by a machine learning algorithm.

17. The computer-implemented method of claim 1, further comprising determining the availability of the one or more staff based on a schedule for each staff and one or more predefined staffing ratios.

18. The computer-implemented method of claim 1, wherein the method is HIPPA compliant.

19. The computer-implemented method of claim 1, further comprising:
transmitting the one or more dates and the time periods to a scheduling system of one or more physicians for treating a secondary condition of the patient; and
determining an overlap between a schedule of the one or more physicians and the one or more dates and the time periods; and scheduling an appointment with the one or more physicians during the one or more dates and the time periods.

20. The computer-implemented method of claim 19, wherein the appointment is a virtual appointment between the patient and the one or more physicians during the dialysis machine usage by the patient.

21. The computer-implemented method of claim 1, further comprising:
receiving a fourth input including historical data comprising appointment frequency per time period for a physician; and automatically updating the one or more dates and the time periods to fall within a window of higher appointment frequency for the physician.

22. A system for optimizing dialysis machine usage, the system comprising:
at least one processor; and
memory storing instructions that, when executed by the at least one processor, cause the system to execute instructions comprising:
receiving a first input indicating a frequency and a duration of dialysis prescribed for a patient in a predefined time window;
receiving a second input indicating an availability of one or more dialysis machines at a location and within the predefined time window;
receiving a third input indicating an availability of one or more staff at the location and within the predefined time window;
determining one or more dates and time periods within the one or more dates in which the patient could receive dialysis based on the frequency and the duration of dialysis, wherein the determined one or more dates and the time periods are aligned with the availability of the one or more dialysis machines and the availability of the one or more staff; and
outputting an indication of the one or more dates and the time periods.

23. The system of claim 22, wherein the instructions further comprise:
receiving a fourth input indicating one or more of: a drive time for the patient, a weather input of the location, and a delay risk metric; and
automatically updating the one or more dates and the time periods based on the fourth input.

24. The system of claim 22, wherein the instructions further comprise:
receiving a fourth input indicating a volume of fluid removed from the patient during a previous time period on a previous date; and
automatically updating the one or more dates and the time periods based on the fourth input.

25. The system of claim 22, wherein the instructions further comprise determining the availability of the one or more staff based on a schedule for each staff and one or more predefined staffing ratios.

26. A computer-readable storage medium storing instructions that, when executed by a computer cause the computer to perform instructions to optimize dialysis machine usage, the instructions comprising:
receiving a first input indicating a frequency and a duration of dialysis prescribed for a patient in a predefined time window;
receiving a second input indicating an availability of one or more dialysis machines at a location and within the predefined time window;
receiving a third input indicating an availability of one or more staff at the location and within the predefined time window;
determining one or more dates and time periods within the one or more dates in which the patient could receive dialysis based on the frequency and the duration of dialysis, wherein the determined one or more dates and the time periods are aligned with the availability of the one or more dialysis machines and the availability of the one or more staff; and
outputting an indication of the one or more dates and the time periods.

27. The computer-readable storage medium of claim 26 wherein the instructions further comprise:
receiving a fourth input indicating one or more of: a drive time for the patient, a weather input of the location, and a delay risk metric; and
automatically updating the one or more dates and the time periods based on the fourth input.

28. The computer-readable storage medium of claim 26, wherein the instructions further comprise receiving a selection input indicating a first time period of the time periods on a first date of the one or more dates.

29. The computer-readable storage medium of claim 26, wherein the instructions further comprise receiving a fourth input indicating one or both of: a fluid load or a lifestyle metric of the patient; and automatically updating the one or more dates and the time periods based on the fourth input.

30. The computer-readable storage medium of claim 29, wherein the fourth input is received from a wearable device.
